(19) United States
(12) Patent Application Publication
(10) Pub. No.: US 2012/0016017 A1
(43) Pub. Date: Jan. 19, 2012

(54) PHARMACEUTICAL COMPOSITION FOR TREATING OR PREVENTING CANCER

(76) Inventor: Gee-Hwoon LEE, Seoul (KR)

(21) Appl. No.: 12/846,303

(22) Filed: Jul. 29, 2010

(30) Foreign Application Priority Data
Jul. 13, 2010 (KR) 10-2010-0067614

(51) Int. Cl. A61K 31/351; A61P 35/00; C07D 309/32

(52) U.S. Cl. 514/459; 549/419

(57) ABSTRACT

The present invention relates to a compound of the formula I:

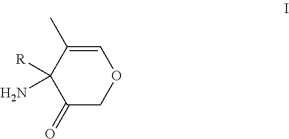

wherein R is C₂H₅ or C₂H₃,
or a pharmaceutically acceptable salt thereof, and a process for preparing said compound of the formula I. The invention also relates to the use of a composition comprising said compound of the formula I or a pharmaceutically acceptable salt thereof as an active ingredient, for treating or preventing cancers.

### CROSS-REFERENCE TO RELATED APPLICATIONS

This application claims the benefit and priority of Korean Application No. 10-2010-0067614, filed Jul. 13, 2010. The entire disclosure of the above application is incorporated herein by reference.

### TECHNICAL FIELD

The present invention relates to a compound of the formula I or a pharmaceutically acceptable salt thereof, and a process for preparing said compound of the formula I. The invention also relates to the use of a composition comprising said compound of the formula I or a pharmaceutically acceptable salt thereof as an active ingredient, for treating or preventing cancers.

### BACKGROUND ART

Cancer is a devastating and debilitating disease that is becoming more prevalent worldwide. Cancer is distinguished by uncontrolled growth and spread of abnormal cells. It can adversely affect all the organs and tissues of the body, often leading to death. Numerous factors play a role in the initiation and progression of cancer, which makes it difficult to cure. The incidences of cancer among younger individuals have also increased in recent years. About 1,444,000 new cases of cancer were diagnosed in the USA in 2007. This does not include noninvasive cancers at any site except urinary bladder, and does not include basal and squamous cell skin cancer. The molecular diagnosis of cancer relies on biomarker molecules that are antigens or proteins expressed at higher levels in cancer cells than normal cells, or are synthesized de novo. These biomarkers are produced directly by tumor cells or by the human body in response to the presence of cancers. Detection of the biomarkers in a patient's sample can serve as an important step in cancer diagnosis. In addition, the ability to screen cancer at an earlier stage increases the survival rate of cancer patients.

Currently there are many types of antitumor agents utilized for cancer treatments. These agents are classified in several different categories. These categories and examples of the most used antitumor agents are described below.

(1) DNA damaging agents: Doxorubicin is an anthracycline antibiotic that works by intercalating into adjacent nucleotides and blocking RNA and DNA synthesis. To accomplish this, it forms tight DNA-drug interactions and also inhibits topoisomerase II, an enzyme essential for DNA synthesis. Metabolism of Doxorubicin produces free oxygen radicals causing peroxidation of lipid membranes and calcium release from the heart tissues, leading to cardiotoxicity. The major clinical problem in Doxorubicin use is drug resistance. In spite of these side effects, Doxorubicin is used for a wide range of cancers and is the most widely used anthracycline.

(2) Alkylating agents: Cyclophosphamide is the most commonly used alkylating agent. Through cytochrome P-450 action, Cyclophosphamide converts to hydroxylated intermediates and forms active phosphoramide mustard and acrolein. Phosphoramide mustard causes interstrand/intrastrand DNA cross-linkage, causing cell death in wide range of cancer cells. Since Cyclophosphamide is carcinogenic, it increases the risk of developing other cancers and suppresses the immune system.

(3) Microtubule inhibitors (MI): MI disrupt spindle microtubule dynamics and cause cell cycle arrest and apoptosis. Taxanes (paclitaxel and docetaxel) are microtubule polymerizing agents and vinca alkaloids are microtubule depolymerizing agents. Taxanes are the most active agents for treating breast cancer. Paclitaxel binds to β-tubulin, causes microtubule' s polymerization and stability, inhibits the metaphase to anaphase transition during mitosis, and induces apoptosis. Docetaxel is a second generation taxane and shares same binding site as paclitaxel with greater affinity. Docetaxel has been shown to have 2 to 4 fold more cytotoxicity than paclitaxel.

(4) Vinca alkaloids: Vincristine, vinblastine, colchicines, podophyllotoxin and nocodazole have high affinity to the ends of microtubules, binding to them and preventing attachment of microtubules to the kinetochores. This causes inhibition of microtubule assembly and destabilizes microtubules leading to apoptosis. They do not share binding sites with taxanes. These MI are generally used as adjuvant therapies to Doxorubicin or Cyclophosphamide treatments.

(5) Aromatase inhibitors (AI): Aromatase coverts androgens into estrogens, thereby increases local estrogen concentrations. This may play an role in breast cancer carcinogenesis. Since Aromatase inhibitors inhibit aromatase but do not block the ovaries from producing estrogen, it only works for post-menopausal women. Aromatase inhibitors can lead to estrogen depletion in the cardiovascular system and bones. Thus, heart problems and osteoporosis are the main side effects.

(6) Non-steroidal hormone therapies: Tamoxifen acts as a selective estrogen receptor modulator (SERM) which exerts antiestrogenic effects by directly binding to the ER α/β and disrupting normal signal transduction in the breast while having estrogenic effects in bone, uterus and cholesterol level, except in patients with ER-negative breast cancers. Its pro-estrogenic effects on the uterus leads to increased chances for development of uterine cancer in breast cancer patients treated with tamoxifen. Raloxifene is next generation of SERM and has anti-estrogenic effects in both breast and uterus. Thus, endometrial growth is not stimulated. Raloxifene was approved by the FDA in 2007 to prevent osteoporosis and risk of invasive breast cancer in postmenopausal women with high risk histories. Raloxifene has pro-estrogen effects in the bone and heart resulting in high density of bones and lowered cholesterol.

All of the above treatment agents have one commonality: they kill cancer and normal cells alike. There are also side effects that greatly decrease the patient's quality of life. There are two promising antitumor agents that have recently been developed. Trastuzumab (Herceptin) is a monoclonal antibody specifically designed to bind to the erbB-2 (her2/neu) receptor. This prevents extracellular growth signals by disrupting ligand and receptor binding, and may induce antibody dependent cellular cytotoxicity. However, Trastuzumab resistance was found at the level of cytoplasmic signal transduction, so additional monoclonal antibodies such as pertuzumab are needed to synergistically block erbB receptor signaling.

Another drug that held great promise was the Tyrosine Kinase Inhibitor GLEEVEC (Imatinib mesilate). Imatinib is a 2-phenylaminopyrimidine derivative that functions as a specific inhibitor of a number of tyrosine kinase enzymes. It functions by occupying the TK active site, leading to a decrease in activity. It is specific for the TK domain in abl (the Abelson protooncogene), c-kit and PDGF-R (platelet-derived growth factor receptor). It works by binding to the ATP binding site of bcr-abl and inhibiting the enzyme activity of the protein competitively. It is selective for bcr-abl, and also inhibits other targets mentioned above (ckit and PDGF-R). However, heart problems, anemia and other side effects in patients treated with GLEEVEC have occurred.

For reasons mentioned above, commercially available antitumor agents have a common problem that if their antitumor effects are enhanced, the resulting high toxicity makes them improper for patients with terminal cancer, the old and children whose body resistance is weak, while if their toxicities are reduced, the desired antitumor effects are not sufficiently obtained. In addition, most of the antitumor agents have side effects such as vomiting, liver toxicity, lung toxicity, neurotoxicity, skin toxicity, hair loss, infertility, and cannot separate between cancer cells and normal cells, and thus they kill cancer cells as well as normal cells. In particular, Paclitaxel, one of the most widely used antitumor agents, is virtually insoluble in water, and thus other substances should be mixed together in order to administer by injection. However, it is reported that the overdose of substances mixed results in cardiotoxicity, hypersensitivity reaction, etc.

Thus, though many ways to kill cancer cells were disclosed in the art, there still remains a need to specifically target cancer cells without toxicities in normal cells, reduce side effects and improve the cytotoxicity of antitumor agents.

### DISCLOSURE

### Technical Problem

It is the object of the present invention to provide a pharmaceutical composition usable to target only abnormal cells such as cancer cells, activate the function of normal cells, repair injured normal cells, have no toxicity, apply to patients with terminal cancer whose body resistance is weak, prepare under the mild condition, have a hydrophile property which makes it much safer in production than that of using organic solvents and available in both injection and oral administration form, and work fast.

### Technical Solution

The present invention relates to a compound of the formula I:

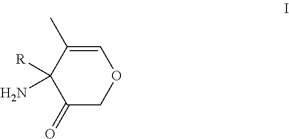

wherein R is C₂H₅ or C₂H₃,

or a pharmaceutically acceptable salt thereof.

The invention also relates to the use of a composition comprising said compound of the formula I or a pharmaceutically acceptable salt thereof as an active ingredient, for treating or preventing cancers. Such cancers can include, but are not limited to, breast cancer, prostate cancer, lung cancer, liver cancer, pancreatic cancer, skin cancer, colorectal cancer, osteosarcoma, brain tumor, etc. The composition according to the invention is effective against all kinds of cancers.

The composition comprising the compound of the formula I or a pharmaceutically acceptable salt thereof according to the invention can comprise pharmaceutically acceptable carriers. And, the composition can additionally comprise other ingredients as long as the activity of the compound of the formula I or a pharmaceutically acceptable salt thereof is not reduced. The pharmaceutically acceptable salts or carriers are well known in the art, and can be selected by person having ordinary skill in the art.

The dose and duration of the treatment will depend on a variety of factors, including the age, body weight, general health, sex, diet and the cancer type of the patient. Preferably, the composition according to the invention can comprise an amount of 0.3 mg or more of compound of the formula I or a pharmaceutically acceptable salt thereof relative to 1 ml of the purified water. Preferably, the composition according to the invention can be administered at single-dose level of 50 ml to 500 ml, and one time to twelve times a day. When administered twelve times a day, it is desirable to be administered every 2 hours.

The composition according to the invention can be administered by all types of route available in the art. For example, the composition according to the invention can be administered by parenteral (e.g. subcutaneously, intramuscularly, intravenously, intraperitoneally, intrapleurally, intravesicularly or intrathecally), topical, oral, rectal, nasal route, etc.

The invention also relates to a process for preparing the compound of the formula I, including:
a) Reacting (i) 2-methyl-butyric acid and (ii) arctigenin-4-O-glucoside, 3,5,7,9-tetrayne or alanine, in a weight ratio of 2:1;
b) Adding (i) copper and (ii) luteolin-7-rhamnoglucoside, saponin, pinene, trans-geraniol, linalol or chlorogenic acid in a weight ratio of 3:1 to the products obtained in the step a), and then reacting;
c) Adding (i) vitexicarpin or hesperidin and (ii) 3′-hydroxyformononetin, kaempferol or water in a weight ratio of 1:1 to the products obtained in the step b), and then reacting;
d) Reacting the products obtained in the step c) and sinigrin; and
e) Reacting the products obtained in the step d) and valine to obtain the compound of the formula I.

In accordance with one embodiment, the reacting in the step a) is carried out at a temperature of 80˜120° C. for 10˜30 minutes, the reacting in the step b) is carried out at 120˜170° C. for 10 minutes, the reacting in the step c) is carried out at 80˜120° C. for 5˜8 minutes, the reacting in the step d) is carried out at 100˜140° C. for 5˜10 minutes, and the reacting in the step e) is carried out at −30˜30° C. for 10˜20 minutes, and then at 80˜230° C. for 5˜30 minutes.

In accordance with one embodiment, water (H₂O) is used as the solvent in the step a) to the step e).

In accordance with one embodiment, the products obtained in the step e) are filtered by water at −5˜30° C.

In accordance with one embodiment, the products obtained in the step e) are filtered by the following steps:
1) Filtering the products obtained in the step e) by water at 100˜150° C.;
2) Filtering the products obtained in the step 1) by water at 70˜100° C.;
3) Filtering the products obtained in the step 2) by water at 40˜60° C.;
4) Filtering the products obtained in the step 3) by water at 15˜30° C.;
5) Filtering the products obtained in the step 4) by water at −1˜15° C.; and
6) Drying the products obtained in the step 5) to obtain the compound of the formula I.

In accordance with one embodiment, the products obtained in the step e) are filtered by the following steps:
1) Filtering the products obtained in the step e) by water at 100˜150° C.;
2) Filtering the products obtained in the step 1) by water at 30˜100° C.;
3) Filtering the products obtained in the step 2) by water at −5˜30° C.; and
4) Drying the products obtained in the step 3) to obtain the compound of the formula I.

Preferably, the filter used in said filtering is the membrane with a pore size of not bigger than 10⁻⁶ m.

When the composition according to the invention is administered into the body, it is found that neurotransmitters are activated, the function of spleen is enhanced, and the blood is purified. Thereby, the composition according to the invention can stimulate the secretion of neurotransmitters and hormones in the body, increase the activity of spleen, and reduce a fever, thus controlling the body's temperature. In addition, it can activate the overall function of lymph, proliferate lymphocytes and macrophagocytes, thus purifying lymph and blood. Furthermore, while most cancer patients can not tolerate conventional antitumor agents having strong toxicity because of weakened body functions and complications, the composition according to the invention can activate the function of normal cells and repair injured normal cells, thereby enhance the weakened body functions of patients, and provide the patient's overall physical condition suitable to be treated with anticancer treatments. Therefore, the composition according to the invention can be used to patients with terminal cancer without causing side effects and drug-shock. And, when the composition according to the invention is administered into the body, it is found that cancer cells stop temporarily their activities, and then the form of cancer cells changes into the polygonal form with black edges, and eventually bursts. As well, the composition according to the invention stimulates marrow, and produces NK and NKT cells. It is differentiated into T and B cells, eliminating cancer cells that blood can reach.

### Advantageous Effects

The composition according to the invention is effective in treating and preventing all kinds of cancers. In particular, unlike conventional antitumor agents to kill both cancer and normal cells, the composition according to the invention can target only abnormal cells such as cancer cells, activate the function of normal cells, and repair injured normal cells. In addition, the composition works very fast and recovers the body's functioning weakened by cancer and complications, and thus is suitable for patients with terminal cancer as well as the old and children. Furthermore, the composition can eliminate latent tumors in the body and enhance immunocyte activity in the blood, thereby prevent cancer in advance.

### DESCRIPTION OF DRAWINGS

The patent or application file contains at least one drawing executed in color. Copies of this patent or patent application publication with color drawing(s) will be provided by the Office upon request and payment of the necessary fee.

In the Figures attached to the specification, the experimental group indicates the group treated with the composition comprising the compound of formula II or formula III, the control group indicates the group treated with Taxol or cisplatin, and the untreated group indicates the group untreated with any agents.

FIG. 1 shows the ¹H NMR spectrum of the compound of the formula II.

FIG. 2 shows the ¹H NMR spectrum of the compound of the formula

FIG. 3 is the graph showing the cell counts determined in cell line HCC 1419 during the 1^{st} week (LC50) and the 2^{nd} week (recovery), respectively. Herein, the horizontal axis represents the time in culture, and the vertical axis represents 1/20 of cell number.

FIGS. 4A and 4B are graphs showing the viability determined in cell line HCC 1419 by XTT assay at a wavelength of 500 nm during the 1st week (LC50) and the 2nd week (recovery), respectively. Herein, the horizontal axis represents the time in culture, and the vertical axis represents 1/100 of viability (%).

FIGS. 5A and 5B are graphs showing the cytotoxicity determined in cell line HCC 1419 by XTT assay at a wavelength of 500 nm during the 1^{st} week (LC50) and the 2^{nd} week (recovery), respectively. Herein, the horizontal axis represents the time in culture, and the vertical axis represents 1/100 of cytotoxicity (%).

FIGS. 6A-C are photographs taken at 48 hours of the 1^{st} week (LC50) in the experimental group, the control group and the untreated group of cell line HCC 1419, respectively. Olympus IX70 inverted microscope, magnifications ×200.

FIGS. 7A-F are photographs taken at 4 days of the 1^{st} week (LC50) in the experimental group, the control group and the untreated group of cell line HCC 1419, respectively. Olympus IX70 inverted microscope, magnifications ×40 (top photographs), ×200 (bottom photographs).

FIGS. 8A-F are photographs taken at 7 days of the 1^{st} week (LC50) in the experimental group, the control group and the untreated group of cell line HCC 1419, respectively. Olympus IX70 inverted microscope, magnifications ×40 (top photographs), ×200 (bottom photographs).

FIGS. 9A and 9B are photographs taken at 7 days of the 1^{st} week (LC50) in the experimental group and the control group of cell line HCC 1419, respectively. Olympus IX70 inverted microscope. The experimental group shows cells constrained to a single colony, whereas the control group shows cells wide-spreading throughout the whole well.

FIGS. 10A and 10B are graphs showing the cell counts determined in cell line MCF-7 during the 1^{st} week (LC50) and the 2^{nd} week (recovery), respectively. Herein, the horizontal axis represents the time in culture, and the vertical axis represents cell number in the 1^{st} week and 1/20 of cell number in the 2^{nd} week.

FIGS. 11A and 11B are graphs showing the cell counts determined in cell line MDA-MB-468 during the 1^{st} week (LC50) and the 2^{nd} week (recovery), respectively. Herein, the horizontal axis represents the time in culture, and the vertical axis represents cell number.

FIG. 12 is the graph showing the cell counts determined in cell line SKBR3 during the 1^{st} week (LC50). Herein, the horizontal axis represents the time in culture, and the vertical axis represents cell number.

FIG. 13 is the graph showing the viability determined by using the trypan blue staining in cell line SKBR3 during the 1^{st} week (LC50). Herein, the horizontal axis represents the time in culture, and the vertical axis represents viability (%).

FIGS. 14A and 14B are graphs showing the cell counts determined in cell line PC3 during the 1st week (LC50) and the 2nd week (recovery), respectively. Herein, the horizontal axis represents the time in culture, and the vertical axis represents 1/20 of cell number.

FIGS. 15A and 15B are graphs showing the viability determined in cell line PC3 by XTT assay at a wavelength of 500 nm during the 1^{st} week (LC50) and the 2^{nd} week (recovery), respectively. Herein, the horizontal axis represents the time in culture, and the vertical axis represents 1/100 of viability (%).

FIGS. 16A and 16B are graphs showing the cytotoxicity determined in cell line PC3 by XTT assay at a wavelength of 500 nm during the 1^{st} week (LC50) and the 2^{nd} week (recovery), respectively. Herein, the horizontal axis represents the time in culture, and the vertical axis represents 1/100 of cytotoxicity (%).

FIGS. 17A and 17B are graphs showing the cell counts determined in cell line HT1299 during the 1st week (LC50) and the 2nd week (recovery), respectively. Herein, the horizontal axis represents the time in culture, and the vertical axis represents 1/20 of cell number.

FIGS. 18A and 18B are graphs showing the viability determined in cell line HT1299 by XTT assay at a wavelength of 500 nm during the 1^{st} week (LC50) and the 2^{nd} week (recovery), respectively. Herein, the horizontal axis represents the time in culture, and the vertical axis represents 1/100 of viability (%).

FIGS. 19A and 19B are graphs showing the cytotoxicity determined in cell line HT1299 by XTT assay at a wavelength of 500 nm during the 1^{st} week (LC50) and the 2^{nd} week (recovery), respectively. Herein, the horizontal axis represents the time in culture, and the vertical axis represents 1/100 of cytotoxicity (%).

FIGS. 20A and 20B are photographs taken at 7 days of the 1st week (LC50) in the experimental group and the control group of cell line HT1299, respectively. Olympus IX70 inverted microscope.

FIGS. 21A-D are photographs taken at 24 hours of the 2^{nd} week (recovery) in the experimental group and the control group of cell line HT1299, respectively. Olympus IX70 inverted microscope.

FIGS. 22A-D are photographs taken at 4 days of the 2^{nd} week (recovery) in the experimental group and the control group of cell line HT1299, respectively. Olympus IX70 inverted microscope.

FIGS. 23A and 23B are graphs showing the cell counts determined in cell line Saos-2 during the 1^{st} week (LC50) and the 2^{nd} week (recovery), respectively. Herein, the horizontal axis represents the time in culture, and the vertical axis represents 1/20 of cell number.

FIGS. 24A and 24B are photographs taken at 24 hours of the 2^{nd} week (recovery) in the experimental group and the control group of cell line Saos-2, respectively. Olympus IX70 inverted microscope.

FIGS. 25A and 25B are photographs taken at 4 days of the 2^{nd} week (recovery) in the experimental group and the control group of cell line Saos-2, respectively. Olympus IX70 inverted microscope.

FIGS. 26A and 26B are photographs taken at 7 days of the 2nd week (recovery) in the experimental group and the control group of cell line Saos-2, respectively. Olympus IX70 inverted microscope.

FIGS. 27A and 27B are graphs showing the cell counts determined in cell line C-6 during the 1st week (LC50). Herein, the horizontal axis represents the time in culture, and the vertical axis represents 1/20 of cell number.

FIG. 28 is the graph showing the cell counts determined in cell line C-6 during the 2nd week (recovery). Herein, the horizontal axis represents the time in culture, and the vertical axis represents 1/20 of cell number.

FIGS. 29A and 29B are graphs showing the viability determined in cell line C-6 by XTT assay at a wavelength of 500 nm during the 1st week (LC50) and the 2nd week (recovery), respectively. Herein, the horizontal axis represents the time in culture, and the vertical axis represents 1/100 of viability (%).

FIGS. 30A and 30B are graphs showing the cytotoxicity determined in cell line C-6 by XTT assay at a wavelength of 500 nm during the 1st week (LC50) and the 2nd week (recovery), respectively. Herein, the horizontal axis represents the time in culture, and the vertical axis represents 1/100 of cytotoxicity (%).

FIGS. 31A and 31B are graphs showing the cell counts determined in cell line AsPc during the 1^{st} week (LC50) and the 2^{nd} week (recovery), respectively. Herein, the horizontal axis represents the time in culture, and the vertical axis represents 1/20 of cell number.

FIGS. 32A and 32B are photographs taken at 7 days of the 1^{st} week (LC50) in the experimental group and the control group of cell line AsPc, respectively. Olympus IX70 inverted microscope.

FIGS. 33A and 33B are photographs taken at 24 hours of the 2^{nd} week (recovery) in the experimental group and the control group of cell line AsPc, respectively. Olympus IX70 inverted microscope.

FIGS. 34A and 34B are photographs taken at 4 days of the 2^{nd} week (recovery) in the experimental group and the control group of cell line AsPc, respectively. Olympus IX70 inverted microscope.

FIGS. 35A and 35B are photographs taken at 7 days of the 2^{nd} week (recovery) in the experimental group and the control group of cell line AsPc, respectively. Olympus IX70 inverted microscope.

FIGS. 36A and 36B are graphs showing the cell counts determined in cell line HME 50 HT during the 1^{st} week (LC50) and the 2^{nd} week (recovery), respectively. Herein, the horizontal axis represents the time in culture, and the vertical axis represents cell number.

FIGS. 37A-F are photographs taken at 48 hours of the 1^{st} week (LC50) in the experimental group and the control group of cell line HME 50 HT, respectively. Olympus IX70 inverted microscope.

FIGS. 38A-F are photographs taken at 7 days of the 1^{st} week (LC50) in the experimental group and the control group of cell line HME 50 HT, respectively. Olympus IX70 inverted microscope.

FIGS. 39A and 39B are photographs taken at 0 days of the 2^{nd} week (recovery) in the experimental group and the control group of cell line HME 50 HT, respectively. Olympus IX70 inverted microscope.

FIGS. 40A and 40B are photographs taken at 7 days of the 2^{nd} week (recovery) in the experimental group and the control group of cell line HME 50 HT, respectively. Olympus IX70 inverted microscope.

FIGS. 41A and 41B are graphs showing the cell counts determined in cell line BJ during the 1^{st} week (LC50) and the 2^{nd} week (recovery), respectively. Herein, the horizontal axis represents the time in culture, and the vertical axis represents cell number in the 1^{st} week and 1/1000 of cell number in the 2^{nd} week.

FIGS. 42A and 42B are graphs showing the viability determined in cell line BJ by XTT assay at a wavelength of 500 nm during the 1^{st} week (LC50) and the 2^{nd} week (recovery), respectively. Herein, the horizontal axis represents the time in culture, and the vertical axis represents 1/100 of viability (%).

FIGS. 43A and 43B are graphs showing the cytotoxicity determined in cell line BJ by XTT assay at a wavelength of 500 nm during the 1^{st} week (LC50) and the 2^{nd} week (recovery), respectively. Herein, the horizontal axis represents the time in culture, and the vertical axis represents 1/100 of cytotoxicity (%).

FIGS. 44A-D are photographs taken at 7 days of the 1^{st} week (LC50) in the experimental group, the control group and the untreated group of cell line BJ, respectively. Olympus IX70 inverted microscope.

FIGS. 45A-D are photographs taken at 4 days of the 2^{nd} week (recovery) in the experimental group and the control group of cell line BJ, respectively. Olympus IX70 inverted microscope.

FIG. 46 is the graph showing the cell counts determined in cell line CCD-1074sk during the 1^{st} week (LC50). Herein, the horizontal axis represents the time in culture, and the vertical axis represents cell number.

FIG. 47 is the graph showing the viability determined by using the trypan blue staining in cell line CCD-1074sk during the 1^{st} week (LC50). Herein, the horizontal axis represents the time in culture, and the vertical axis represents viability (%).

FIG. 48 is the graph showing the cell counts determined in cell line CCD-1074sk during the 2^{nd} week (recovery). Herein, the horizontal axis represents the time in culture, and the vertical axis represents cell number.

FIG. 49 is the graph showing the viability determined by using the trypan blue staining in cell line CCD-1074sk during the 2^{nd} week (recovery). Herein, the horizontal axis represents the time in culture, and the vertical axis represents viability (%).

FIG. 50 is the graph showing the size of tumor determined in the untreated group, the experimental group and the control group (cisplatin) after 5-week-old female nude mice (BALB/c nu/nu) were inoculated with WM-266-4 (human melanoma).

FIG. 51 is the graph showing body weight changes in male and female Sprague-Dawley rats after the composition according to the invention is administered.

FIGS. 52A and 52B show the photographs taken at 4 days of the 1^{st} week (LC50) in the experimental group and the control group of cell line HME 50 HT, while FIGS. 52C and 52D show the photographs taken at 4 days of the 1^{st} week (LC50) in the experimental group and the control group of cell line BJ.

FIGS. 53A-D are photographs showing that hairs started to grow on heads and flanks after the composition according to the invention was administered to 5-week-old female nude mice (BALB/c nu/nu).

FIGS. 54A and 54B show the photographs taken at 7 days of the 1^{st} week (LC50) in the experimental group and the control group of cell line HT1299, while FIGS. 54C and 54D show the photographs taken at 48 hours of the 2^{nd} week (recovery) in the experimental group and the control group of cell line AsPc.

### EXAMPLES

The present invention is described in further detail in the following Examples which are not in any way intended to limit the scope of the invention as claimed. In addition, it will appear to those ordinarily skilled in the art that various modifications may be made to the disclosed embodiments, and that such modifications are intended to be within the scope of the present invention.

### Example 1

2-methyl-butyric acid and alanine in a weight ratio of 2:1 were reacted at 120° C. and 3.0 atm for 30 minutes. After that, copper and chlorogenic acid in a weight ratio of 3:1 were added to the products obtained by said reaction, and were then reacted at 120˜170° C. and 2.6˜3.0 atm for 10 minutes. After that, hesperidin and water in a weight ratio of 1:1 were added to the products obtained by said reaction, and were then reacted at 80˜120° C. and 2.7˜3.5 atm for 8 minutes. After that, sinigrin was added to the products obtained by said reaction, and was then reacted at 100° C. and 2.0 atm for 10 minutes. After that, valine was added to the products obtained by said reaction, and was then reacted at −10˜30° C. and 2˜7 atm for 10 minutes, and in succession was reacted at 200˜230° C. and 1 atm for 10 minutes. In all of the reaction steps, water was used as the solvent.

The final resultant products were purified by two different processes respectively.

As the first purifying process, the final resultant products were dissolved in water at 115˜125° C., and then filtered using the 25 mm nylon syringe filter with 0.2 μm pore size, purchased from VWR. Then, a filtration was successionally performed using the same filter at the temperature of 75˜85° C., 55˜65° C., 27˜33° C. and 2˜22° C., respectively. After that, the filtered solution was vacuum-dried to obtain solid compounds.

As another purifying process, the final resultant products were dissolved in water at 110˜130° C., and then filtered using the 25 mm nylon syringe filter with 0.2 μm pore size, purchased from VWR. Then, a filtration was successionally performed using the same filter at the temperature of 70˜90° C. and 23˜27° C., respectively. After that, the filtered solution was vacuum-dried to obtain solid compounds.

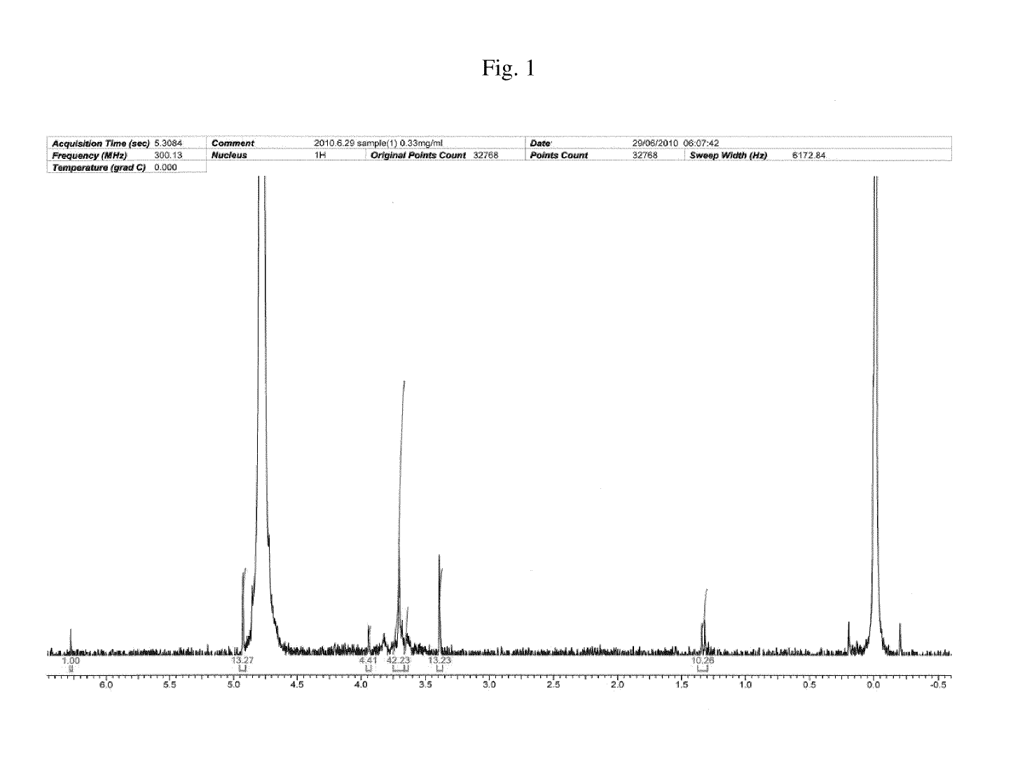

Both compounds obtained by said two processes show the 1H NMR spectrum of FIG. 1, and are determined to have the following formula II:

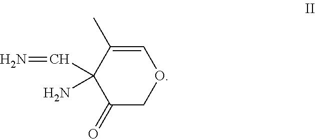

### Example 2

2-methyl-butyric acid and alanine in a weight ratio of 2:1 were reacted at 80˜120° C. and 2.7˜3.0 atm for 10 minutes. After that, copper and chlorogenic acid in a weight ratio of 3:1 were added to the products obtained by said reaction, and were then reacted at 120˜170° C. and 2.6˜3.0 atm for 10 minutes. After that, hesperidin and water in a weight ratio of 1:1 were added to the products obtained by said reaction, and were then reacted at 80˜120° C. and 2.7˜3.5 atm for 8 minutes. After that, sinigrin was added to the products obtained by said reaction, and was then reacted at 120˜140° C. and 3.0˜5.0 atm for 5 minutes. After that, valine was added to the products obtained by said reaction, and was then reacted at −10˜30° C. and 2˜7 atm for 10 minutes, and in succession was reacted at 80˜130° C. and 2˜7 atm for 5 minutes. In all of the reaction steps, water was used as the solvent.

The final resultant products were purified by the two kinds of processes respectively, same as in the Example 1.

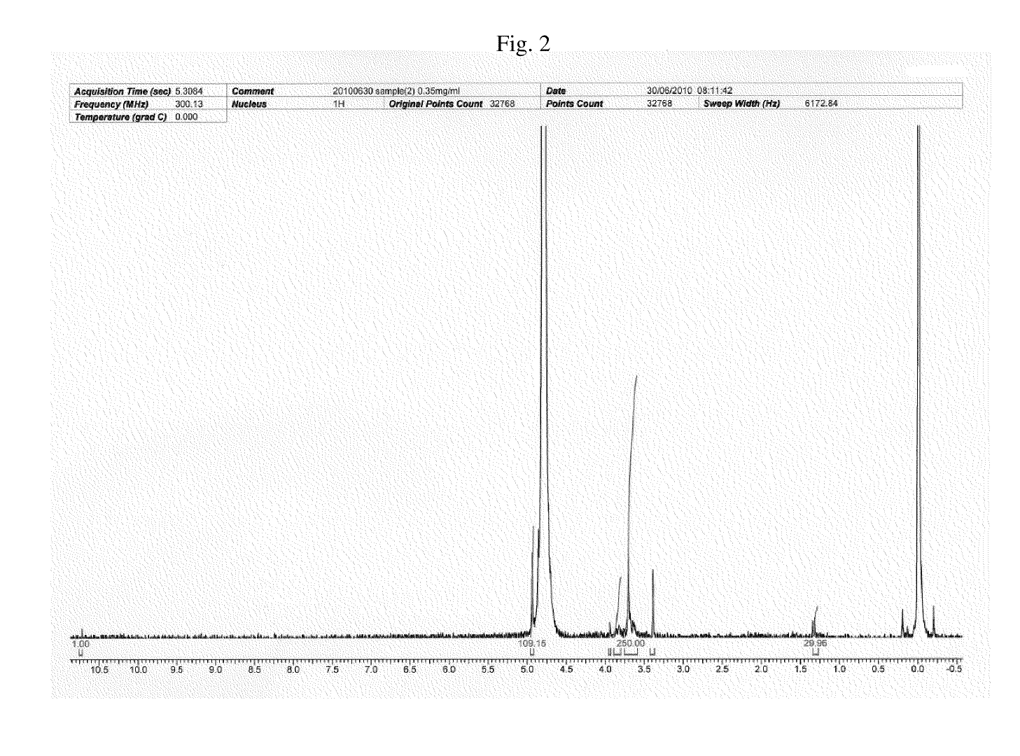

Both compounds obtained by said two processes show the ¹H NMR spectrum of FIG. 2, and are determined to have the following formula III:

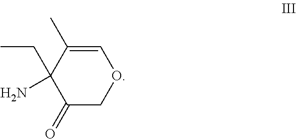

### Example 3

2-methyl-butyric acid and alanine in a weight ratio of 2:1 were reacted at 80˜120° C. and 2.7˜3.0 atm for 10 minutes. After that, copper and chlorogenic acid in a weight ratio of 3:1 were added to the products obtained by said reaction, and were then reacted at 120˜170° C. and 2.6˜3.0 atm for 10 minutes. After that, hesperidin and kaempferol in a weight ratio of 1:1 were added to the products obtained by said reaction, and were then reacted at 80˜120° C. and 1.3 atm for 8 minutes. After that, sinigrin was added to the products obtained by said reaction, and was then reacted at 120˜140° C. and 3.0˜5.0 atm for 5 minutes. After that, valine was added to the products obtained by said reaction, and was then reacted at −10˜30° C. and 2˜7 atm for 10 minutes, and in succession was reacted at 80˜130° C. and 2˜7 atm for 5 minutes. In all of the reaction steps, water was used as the solvent.

The final resultant products were purified by the two kinds of processes respectively, same as in the Example 1. Both compounds obtained by said two processes show the ¹H NMR spectrum of FIG. 2, and are determined to have the formula III.

### Example 4

2-methyl-butyric acid and alanine in a weight ratio of 2:1 were reacted at 80˜120° C. and 2.7˜3.0 atm for 10 minutes. After that, copper and chlorogenic acid in a weight ratio of 3:1 were added to the products obtained by said reaction, and were then reacted at 120˜170° C. and 2.6˜3.0 atm for 10 minutes. After that, hesperidin and 3′-hydroxyformononetin in a weight ratio of 1:1 were added to the products obtained by said reaction, and were then reacted at 120° C. and 2.7 atm for 5 minutes. After that, sinigrin was added to the products obtained by said reaction, and was then reacted at 120˜140° C. and 3.0˜5.0 atm for 5 minutes. After that, valine was added to the products obtained by said reaction, and was then reacted at −10˜30° C. and 2˜7 atm for 10 minutes, and in succession was reacted at 80˜130° C. and 2˜7 atm for 5 minutes. In all of the reaction steps, water was used as the solvent.

The final resultant products were purified by the two kinds of processes respectively, same as in the Example 1. Both compounds obtained by said two processes show the 1H NMR spectrum of FIG. 2, and are determined to have the formula III.

### Example 5

2-methyl-butyric acid and arctigenin-4-O-glucoside in a weight ratio of 2:1 were reacted at 80˜120° C. and 2.7˜3.0 atm for 10 minutes. After that, copper and luteolin-7-rhamnoglucoside in a weight ratio of 3:1 were added to the products obtained by said reaction, and were then reacted at 120˜170° C. and 2.7 atm for 10 minutes. After that, vitexicarpin and 3′-hydroxyformononetin in a weight ratio of 1:1 were added to the products obtained by said reaction, and were then reacted at 120° C. and 2.7 atm for 5 minutes. After that, sinigrin was added to the products obtained by said reaction, and was then reacted at 120˜140° C. and 3.0˜5.0 atm for 5 minutes. After that, valine was added to the products obtained by said reaction, and was then reacted at −10˜30° C. and 2˜7 atm for 10 minutes, and in succession was reacted at 80˜130° C. and 2˜7 atm for 5 minutes. In all of the reaction steps, water was used as the solvent.

The final resultant products were purified by the two kinds of processes respectively, same as in the Example 1. Both compounds obtained by said two processes show the ¹H NMR spectrum of FIG. 2, and are determined to have the formula III.

### Example 6

### In Vitro Experiments

In the following, the values for the composition according to the invention indicate the mean value of values obtained using the composition comprising the compound of formula II and of formula III.

Cancer cell lines HCC 1419, MCF-7, MDA-MB-468, SKBR3, PC3, HT1299, Saos-2, C-6 and AsPc, and normal cell lines HME 50 HT, BJ and CCD-1074sk were tested for changes in metabolism (XTT assay) and cell death (cell counts). Each of the cell lines was cultured in the medium shown in the following Table 1.

TABLE 1

| Cell lines | Medium formulation |
|---|---|
| HCC 1419 | DMEM plus 10% fetal bovine serum |
| MCF-7 | DMEM plus 10% fetal bovine serum |
| MDA-MB-468 | DMEM plus 10% fetal bovine serum |
| SKBR3 | McCoy's 5a Medium Modified plus 10% fetal bovine |
| | serum |
| PC3 | Ham's F12K plus 10% fetal bovine serum |
| HT1299 | DMEM plus 10% fetal bovine serum |
| Saos-2 | McCoy's 5a Medium Modified plus 15% fetal bovine |
| | serum |
| C-6 | RPMI 1640 plus 10% fetal bovine serum |
| AsPc | RPMI 1640 plus 10% fetal bovine serum |
| HME 50 HT | Serum-free medium (SF-171 from Clonetics Corp., San |
| | Diego, Calif.) |
| BJ | DMEM plus 10% fetal bovine serum |
| CCD-1074sk | IMDM plus 10% fetal bovine serum |

*DMEM = Dulbecco's Minimum Essential Medium; IMDM = Iscove's Modified Dulbecco's Medium; RPMI = Roswell Park Memorial Institute medium

Cells were plated into 48-well Corning CellBind™ plates at densities of 20,000 cells for normal cells and 10,000˜20,000 cells for cancer cells. 24 to 48 h after plating, cells were treated with the composition according to the invention at concentrations of 4.5e-2M. The first time point treated with the composition of the invention is 0 h. At 3 days after the first treatment with the composition of the invention, medium was changed and the composition of the invention at the same concentrations was added to the cultured cells.

Concurrently, Taxol at concentrations of 2.2e-7M for 24 h were added to parallel cultures of cells 24˜48 h after plating as the positive control group, whereas medium alone was used as the negative control group.

Cell Counts:

Cells were harvested using Trypsin-EDTA at the designated time points. When total cells for each well were counted with a Beckman-Coulter Z1 Particle Characterization Unit, the vertical axis represents 1/20 of cell number in the Figures attached to the specification, while when counted with a Beckman-Coulter Vi-Cell, the vertical axis represents cell number in the Figures. Two 48-well plates were used per cell line, and they were measured at 0 h, 12 h, 24 h, 48 h, 4 d and 7 d, respectively.

In the 1^{st} plate, cells were treated with the composition according to the invention and medium was changed at 0 h and 3 d. Cells treated with Taxol were exposed for 24 h, and then medium was replaced with untreated medium. As a result, LC-50 curves were generated in the 1^{st} week.

In the 2nd plate, 7 d of the 1^{st} week (LC-50) is the same as 0 h of the 2^{nd} week (recovery).

At 0 h of the 2^{nd} week, medium was exchanged with untreated medium for the remainder of the experiment. The untreated controls were not measured in the 2^{nd} week (recovery) due to overconfluence by the end of the 1^{st} week. As a result, Recovery curves were generated in the 2^{nd} week.

The XTT Cell Viability Assay:

The XTT assay is an accepted analysis technique for viability and cytotoxicity of anticancer drugs or other pharmaceutical compositions. Cells were seeded in a 96-well tissue culture plate at a density of 10,000 cells for normal cells and 5,000 cells for cancer cells. After incubation period, the formazan dye formed was quantitated using an ELISA reader. The optimal wavelength used in experiments was 500 nm, and it was measured at 0 h, 12 h, 24 h, 48 h, 4 d and 7 d, respectively. The obtained values were generated using the software SoftMax Pro 4.8.

Determining Cytotoxicity by XTT Assay:

The cell viability was calculated according to the manufacturer's instructions as follows (the term “blank” means XTT in medium only):

[Treated (the composition of the invention or Taxol)−blank]/Control (untreated)×100%

Cellular cytotoxicity was calculated according to the manufacturer's instructions as follows:

[Control (untreated)−(Treated (the composition of the invention or Taxol)−blank)]/Control (untreated)×100%

The experimental group treated with the composition of the invention, the control group treated with Taxol, and the untreated group were treated and measured in the same manner.

(1) HCC 1419

HCC 1419 is a primary ductal carcinoma cell line. The cells are poorly differentiated, overexpressing Her2-neu, negative for p53 expression. HCC1419 is positive for the epithelial cell specific marker, epithelial glycoprotein 2 (EGP2) and for cytokeratin 19. The cells are negative for estrogen receptor and progesterone receptor.

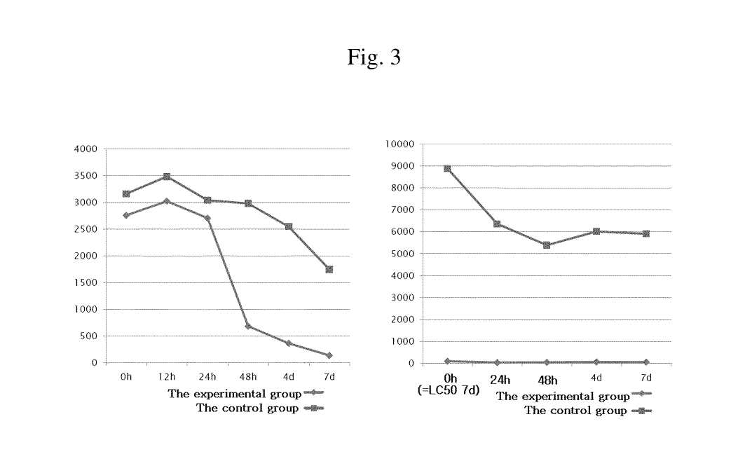

The following Table shows the cell counts determined in cell line HCC 1419 during the 1^{st} week (LC50) (FIG. 3).

TABLE 2

| | 0 h | 12 h | 24 h | 48 h | 4 d | 7 d |
|---|---|---|---|---|---|---|
| The | 2727 | 3057 | 2782 | 847 | 451 | 152 |
| experimental | 3142 | 3096 | 2672 | 624 | 404 | 113 |
| group | 2392 | 2906 | 2651 | 573 | 227 | 133 |
| Mean value | 2754 | 3020 | 2702 | 681 | 361 | 133 |
| The control | 2626 | 3451 | 3488 | 3502 | 2940 | 1710 |
| group (Taxol) | 3788 | 3553 | 2664 | 3199 | 2975 | 1794 |
| | 3055 | 3434 | 2957 | 2232 | 1716 | 1718 |
| Mean value | 3156 | 3479 | 3036 | 2978 | 2544 | 1741 |
| The untreated | 2633 | 2717 | 6776 | 6995 | 25178 | 15380 |
| group | 2954 | 5101 | 6256 | 5943 | 23320 | 10355 |
| Mean value | 2794 | 3909 | 6516 | 6469 | 24249 | 12868 |

The following Table shows the cell counts determined in cell line HCC 1419 during the 2^{nd} week (recovery) (FIG. 3).

TABLE 3

| | 0 h | 24 h | 48 h | 4 d | 7 d |
|---|---|---|---|---|---|
| The experimental | 69 | 57 | 81 | 52 | 32 |
| group | 122 | 28 | 14 | 29 | 58 |
| | 89 | 18 | 40 | 107 | 75 |
| Mean value | 93 | 34 | 45 | 63 | 55 |
| The control | 8522 | 5340 | 4901 | 5721 | 7316 |
| group (Taxol) | 9639 | 7966 | 5431 | 6317 | 3792 |
| | 8472 | 5755 | 5830 | 6001 | 6604 |
| Mean value | 8878 | 6354 | 5387 | 6013 | 5904 |

From the above data, it is clear that the number of cancer cells decreased sharply and did not recover in the experimental group, whereas the number did not decreased any more and was maintained in the control group (Taxol).

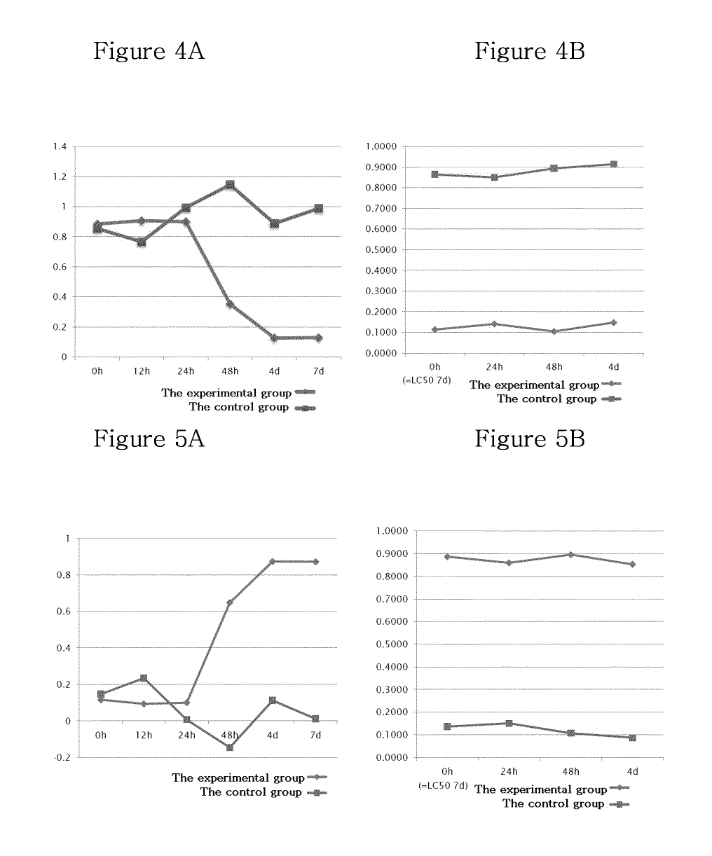

The following Table shows the absorbance, the viability and the cytotoxicity determined in cell line HCC 1419 during the 1^{st} week (LC50) (FIGS. 4 and 5).

TABLE 4

| | | | | | | |
|---|---|---|---|---|---|---|
| Blank | 0 h | 12 h | 24 h | 48 h | 4 d | 7 d |

The absorbance

| | | | | | | | |
|---|---|---|---|---|---|---|---|
| The | 0.3091 | 2.6948 | 2.3571 | 2.3427 | 0.6604 | 0.5811 | 0.5484 |
| experimental | 0.3244 | 2.6082 | 2.8558 | 2.5292 | 1.7252 | 0.8031 | 0.5728 |
| group | 0.3257 | 2.6682 | 2.9637 | 2.8928 | 1.4631 | 0.5696 | 0.6947 |
| Mean value | 0.3197 | 2.6571 | 2.7255 | 2.5882 | 1.2829 | 0.6513 | 0.6053 |
| The control | 0.3578 | 2.5679 | 2.3916 | 2.7216 | 3.7087 | 2.6316 | 2.3859 |
| group (Taxol) | 0.3241 | 2.5534 | 2.3919 | 2.9427 | 3.7945 | 2.6667 | 2.6877 |
| | 0.328 | 2.6558 | 2.3215 | 2.856 | 2.9086 | 2.7014 | 2.5421 |
| Mean value | 0.3366 | 2.5924 | 2.3683 | 2.8401 | 3.4706 | 2.6666 | 2.5386 |
| The untreated | 0.3293 | 2.6463 | 2.5668 | 2.4527 | 2.7876 | 2.5978 | 2.3694 |
| group | 0.3752 | 2.6366 | 2.739 | 2.5872 | 2.6812 | 2.649 | 2.0864 |
| Mean value | 0.3523 | 2.6415 | 2.6529 | 2.5200 | 2.7344 | 2.6234 | 2.2279 |
| The | | 2.3373 | 2.4058 | 2.2685 | 0.9632 | 0.3315 | 0.2856 |
| experimental | | | | | | | |
| group - Blank | | | | | | | |
| The control | | 2.2557 | 2.0317 | 2.5035 | 3.1340 | 2.3299 | 2.2019 |
| group (Taxol) - | | | | | | | |
| Blank | | | | | | | |

The viability

| | | | | | | |
|---|---|---|---|---|---|---|
| The | 0.8849 | 0.9069 | 0.9002 | 0.3522 | 0.1264 | 0.1282 |
| experimental | | | | | | |
| group | | | | | | |
| The control | 0.8540 | 0.7658 | 0.9935 | 1.1461 | 0.8881 | 0.9883 |
| group (Taxol) | | | | | | |

The cytotoxicity

| | | | | | | |
|---|---|---|---|---|---|---|
| The | 0.1151 | 0.0931 | 0.0998 | 0.6478 | 0.8736 | 0.8718 |
| experimental | | | | | | |
| group | | | | | | |
| The control | 0.1460 | 0.2342 | 0.0065 | −0.1461 | 0.1119 | 0.0117 |
| group (Taxol) | | | | | | |

The following Table shows the absorbance, the viability and the cytotoxicity determined in cell line HCC 1419 during the 2^{nd} week (recovery) (FIGS. 4 and 5).

TABLE 5

| | | | | |
|---|---|---|---|---|
| Blank | 0 h | 24 h | 48 h | 4 d |

The absorbance

| | | | | | |
|---|---|---|---|---|---|
| The experimental | 0.2583 | 0.5797 | 0.5209 | 0.62 | 0.7201 |
| group | 0.2386 | 0.5556 | 0.7328 | 0.5621 | 0.641 |
| | 0.3778 | 0.6385 | 0.6853 | 0.5474 | 0.6722 |
| Mean value | 0.2916 | 0.5913 | 0.6463 | 0.5765 | 0.6778 |
| The control group | 0.2348 | 2.3974 | 2.361 | 2.6588 | 2.6116 |
| (Taxol) | 0.1947 | 2.7348 | 2.2497 | 2.6611 | 2.7024 |
| | 0.197 | 2.3412 | 2.4388 | 2.6375 | 2.5033 |
| Mean value | 0.2088 | 2.4911 | 2.3498 | 2.6525 | 2.6058 |
| The untreated | | | | | |
| group | | | | | |
| Mean value | 0.3366 | 2.6415 | 2.52 | 2.7344 | 2.6234 |
| The experimental | | 0.2997 | 0.3548 | 0.2849 | 0.3862 |
| group—Blank | | | | | |
| The control group | | 2.2823 | 2.1410 | 2.4436 | 2.3969 |
| (Taxol)—Blank | | | | | |

The viability

| | | | | |
|---|---|---|---|---|
| The | 0.1135 | 0.1408 | 0.1042 | 0.1472 |
| experimental | | | | |
| group | | | | |
| The control | 0.8640 | 0.8496 | 0.8937 | 0.9137 |
| group (Taxol) | | | | |

The cytotoxicity

| | | | | |
|---|---|---|---|---|
| The | 0.8865 | 0.8592 | 0.8958 | 0.8528 |
| experimental | | | | |
| group | | | | |
| The control | 0.1360 | 0.1504 | 0.1063 | 0.0863 |
| group (Taxol) | | | | |

From the above data, it is clear that the viability of cancer cells is close to 0 in the experimental group, and the cancer cells are barely present in the well.

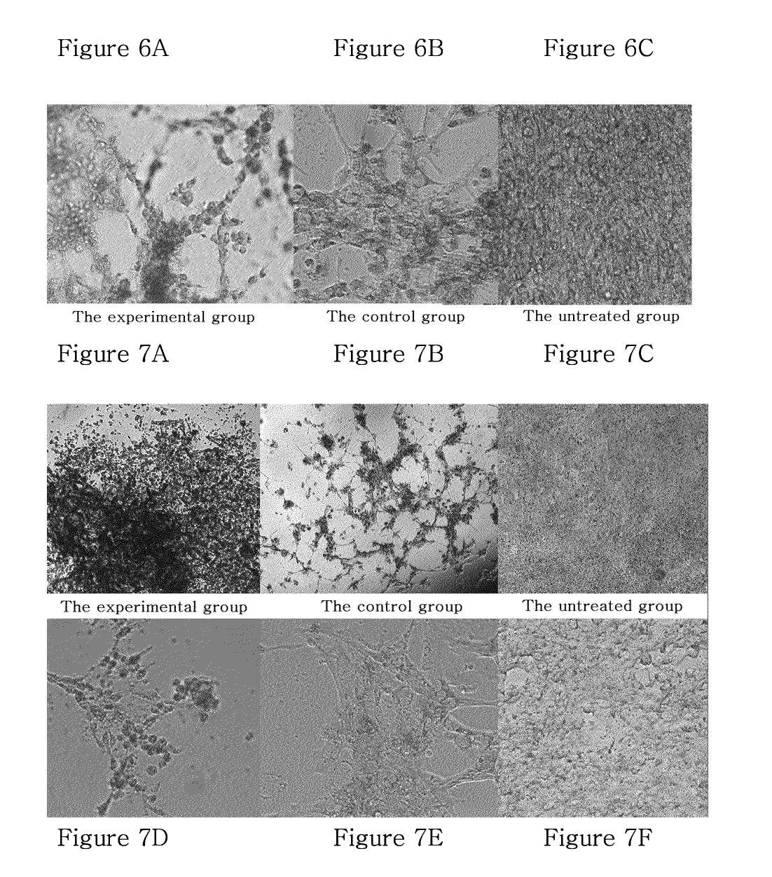

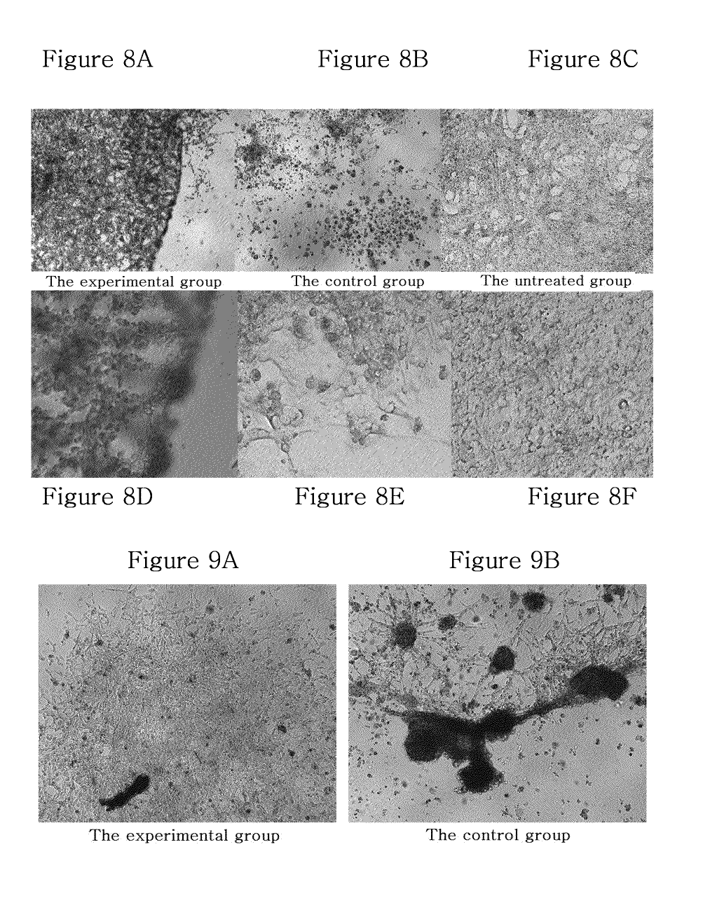

In addition, when performing the analysis of HCC 1419, it was observed that the cells treated with the composition of the invention were constrained to a single colony, lumping together, taking a form like a sheet of paper, and did not grow or spread throughout the whole well. All of these cells were dead ones, and came away from the plate when the well was rinsed for cell counting. However, the cells treated with Taxol were growing in a diffuse, spreading pattern over the whole well. This means that the composition according to the invention can inhibit or change the metastatic potential of cancer cells (FIGS. 6 to 9).

(2) MCF-7

MCF-7 is a breast cancer cell line, and expresses all 3 isoforms of the estrogen receptor. Its growth can be modulated through these estrogen receptor.

During the 1^{st} week (LC50), it was observed that the composition of the invention did not result in dramatic cell death as compared to Taxol. However, during the 2^{nd} week (recovery), the total cell population treated with the composition of the invention died, and no further cell recovery was observed. This is in contrast to Taxol, which showed a significant recovery and maintained a small cell population.

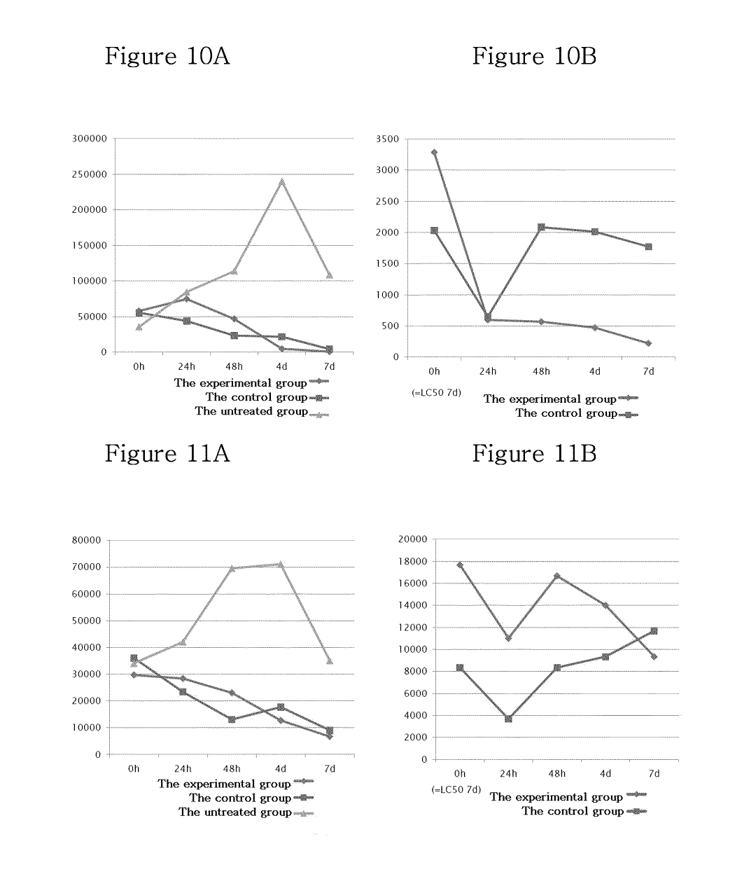

The following Table shows the cell counts determined in cell line MCF-7 during the 1^{st} week (LC50) (FIG. 10).

TABLE 6

| | 0 h | 24 h | 48 h | 4 d | 7 d |
|---|---|---|---|---|---|
| The | 45000 | 68000 | 120000 | 2000 | 0 |
| experimental | 66000 | 56000 | 13000 | 4000 | 0 |
| group | 62000 | 100000 | 7000 | 8000 | 2000 |
| Mean value | 57667 | 74667 | 46667 | 4667 | 667 |
| The control | 67000 | 54000 | 19000 | 7000 | 4000 |
| group (Taxol) | 60000 | 38000 | 30000 | 27000 | 5000 |
| | 39000 | 39000 | 21000 | 31000 | 4000 |
| Mean value | 55333 | 43667 | 23333 | 21667 | 4333 |
| The untreated | 34000 | 88000 | 150000 | 250000 | 120000 |
| group | 37000 | 81000 | 78000 | 230000 | 97000 |
| Mean value | 35500 | 84500 | 114000 | 240000 | 108500 |

The following Table shows the cell counts determined in cell line MCF-7 during the 2^{nd} week (recovery) (FIG. 10).

TABLE 7

| | 0 h | 24 h | 48 h | 4 d | 7 d |
|---|---|---|---|---|---|
| The experimental | 3181 | 563 | 652 | 325 | 271 |
| group | 4035 | 702 | 461 | 417 | 151 |
| | 2646 | 517 | 581 | 666 | 234 |
| Mean value | 3287 | 594 | 565 | 469 | 219 |
| The control | 1648 | 807 | 2010 | 1892 | 1693 |
| group (Taxol) | 2087 | 728 | 2517 | 2011 | 1524 |
| | 2361 | 404 | 1726 | 2135 | 2098 |
| Mean value | 2032 | 646 | 2084 | 2013 | 1772 |

(3) MDA-MB-468

MDA-MB-468 is derived from tissue of patients with metastatic adenocarcinoma. EGF receptor is present at 1×10⁶ per this cell.

In the case of MDA-MB-468, there were few cells left in the wells, and the left cells looked like they were either very stressed or dead. In comparing the results of the experimental group to the control group, it is clear that the composition of the invention was negatively impacting the cells, whereas the cells treated with Taxol were recovering. These observations are confirmed by the fact that cell counts were decreasing in the experimental group, whereas they were increasing in the control group (Taxol). Furthermore, given the fact that the composition of the invention does not show any cytotoxicity in normal cells, it is conceivable that a higher concentration of the composition of the invention or a treatment for a longer time period would show greater killing capacity.

The following Table shows the cell counts determined in cell line MDA-MB-468 during the 1^{st} week (LC50) (FIG. 11).

TABLE 8

| | 0 h | 24 h | 48 h | 4 d | 7 d |
|---|---|---|---|---|---|
| The | 32000 | 39000 | 21000 | 10000 | 6000 |
| experimental | 33000 | 21000 | 25000 | 13000 | 1000 |
| group | 24000 | 25000 | | 15000 | 13000 |
| Mean value | 29667 | 28333 | 23000 | 12667 | 6667 |
| The control | 32000 | 30000 | 12000 | 18000 | 7000 |
| group (Taxol) | 40000 | 16000 | 17000 | 20000 | 3000 |
| | | 24000 | 10000 | 15000 | 17000 |
| Mean value | 36000 | 23333 | 13000 | 17667 | 9000 |
| The untreated | 38000 | 45000 | 61000 | 82000 | 23000 |
| group | 30000 | 39000 | 78000 | 60000 | 47000 |
| Mean value | 34000 | 42000 | 69500 | 71000 | 35000 |

The following Table shows the cell counts determined in cell line MDA-MB-468 during the 2^{nd} week (recovery) (FIG. 11).

TABLE 9

| | 0 h | 24 h | 48 h | 4 d | 7 d |
|---|---|---|---|---|---|
| The | 17000 | 16000 | 20000 | 20000 | 4000 |
| experimental | 23000 | 8000 | 10000 | 18000 | 9000 |
| group | 13000 | 9000 | 20000 | 4000 | 15000 |
| Mean value | 17667 | 11000 | 16667 | 14000 | 9333 |
| The control | 12000 | 3000 | 4000 | 10000 | 11000 |
| group (Taxol) | 9000 | 4000 | 4000 | 9000 | 15000 |
| | 4000 | 4000 | 17000 | 9000 | 9000 |
| Mean value | 8333 | 3667 | 8333 | 9333 | 11667 |

From the above data, it is clear that numbers of cancer cells were continuously decreasing in the experimental group, whereas they were again recovering in the control group (Taxol).

(4) SKBR3

SKBR3 is derived from patients with metastatic pleural effusion.

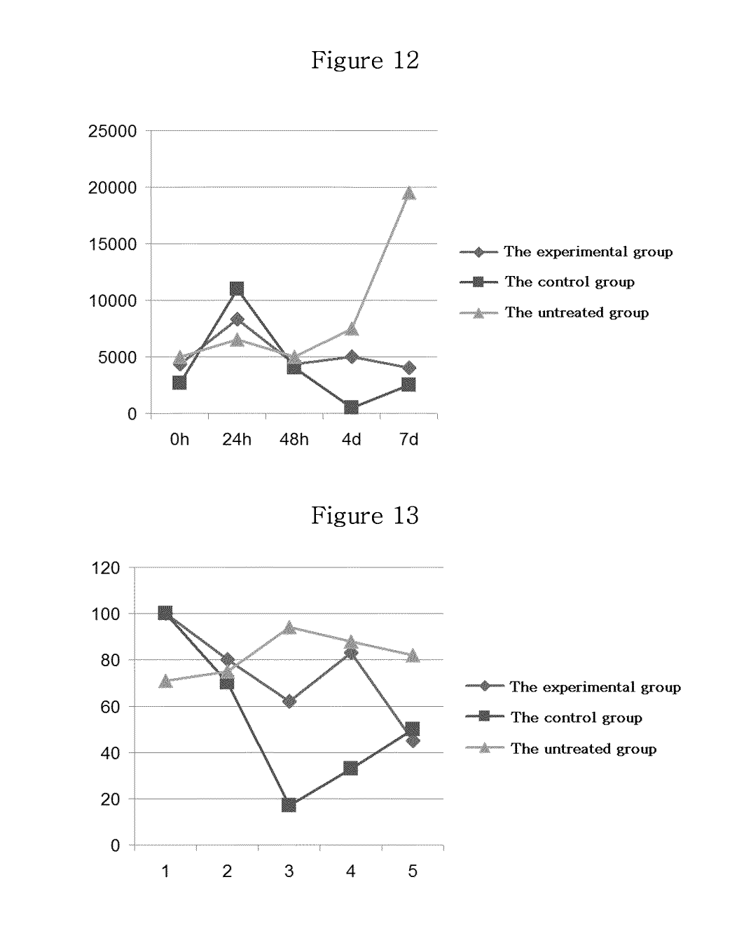

The cell counts is shown in the following Table 10 and the viability is shown in the following Table 11, after determined in cell line SKBR3 during the 1^{st} week (LC50) (FIGS. 12 and 13). The viability was determined by using the trypan blue staining in the well of cell counting.

TABLE 10

| | 0 h | 24 h | 48 h | 4 d | 7 d |
|---|---|---|---|---|---|
| The experimental | 4000 | 12000 | 5000 | 4000 | 4000 |
| group | 5000 | 8000 | 4000 | 3000 | 4000 |
| | 4000 | 5000 | 4000 | 8000 | 4000 |
| Mean value | 4333 | 8333 | 4333 | 5000 | 4000 |
| The control | 2000 | 11000 | 0 | 0 | 1000 |
| group (Taxol) | 4000 | 11000 | 0 | 1000 | 4000 |
| | 2000 | 6000 | 4000 | 0 | 4000 |
| Mean value | 2667 | 11000 | 0 | 500 | 2500 |
| The untreated | 7000 | 7000 | 4000 | 6000 | 12000 |
| group | 3000 | 6000 | 6000 | 9000 | 27000 |
| Mean value | 5000 | 6500 | 5000 | 7500 | 19500 |

TABLE 11

| | 0 h | 24 h | 48 h | 4 d | 7 d |
|---|---|---|---|---|---|
| The experimental | 100 | 79 | 67 | 100 | 60 |
| group | 100 | 78 | 80 | 100 | 50 |
| | 100 | 83 | 40 | 50 | 25 |
| Mean value | 100 | 80 | 62 | 83 | 45 |
| The control | 100 | 69 | 0 | 0 | 100 |
| group (Taxol) | 100 | 69 | 0 | 100 | 50 |
| | 100 | 71 | 50 | 0 | 0 |
| Mean value | 100 | 70 | 17 | 33 | 50 |
| The untreated | 75 | 63 | 100 | 86 | 86 |
| group | 67 | 86 | 88 | 90 | 77 |
| Mean value | 71 | 75 | 94 | 88 | 82 |

(5) PC-3

PC-3 cells were isolated from a bone metastasis of a prostatic adenocarcinoma. The cells exhibit low acid phosphatase and testosterone-5-alpha reductase activities. PC-3 human prostate cancer cell lines are the classical cell lines of prostatic cancer, have high metastatic potential, and do not express p53 or p63. Although this and other studies showed that PC-3 cells were relatively resistant to Taxol, the composition according to the invention was very effective at killing PC-3 cells. Nor was there significant recovery noted.

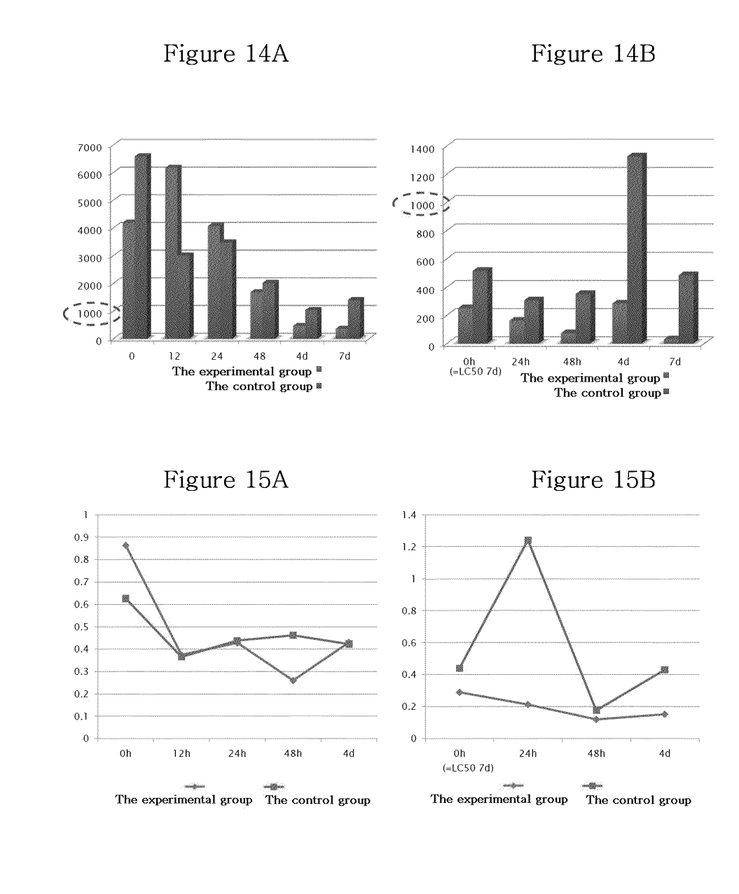

The following Table shows the cell counts determined in cell line PC-3 during the 1^{st} week (LC50) (FIG. 14).

TABLE 12

| | 0 h | 12 h | 24 h | 48 h | 4 d | 7 d |
|---|---|---|---|---|---|---|
| The | 4285 | 4650 | 5555 | 1994 | 491 | 371 |
| experimental | 3200 | 5920 | 7330 | 1937 | 345 | 535 |
| group | 5075 | 5755 | 5570 | 1097 | 536 | 159 |
| Mean value | 4187 | 5442 | 6152 | 1676 | 457 | 355 |
| The control | 5445 | 5805 | 4820 | 1969 | 975 | 1796 |
| group (Taxol) | 9845 | 5775 | 6435 | 2248 | 1250 | 1542 |
| | 4450 | 4805 | 4360 | 1821 | 861 | 829 |
| Mean value | 6580 | 5462 | 5205 | 2013 | 1029 | 1389 |
| The untreated | 5550 | 11870 | 10020 | 13280 | 6175 | 2275 |
| group | 2665 | 8580 | 8245 | 13601 | 6172 | 4649 |
| Mean value | 4108 | 10225 | 9132.5 | 13440.5 | 6173.5 | 3462 |

The following Table shows the cell counts determined in cell line PC-3 during the 2^{nd} week (recovery) (FIG. 14).

TABLE 13

| | 0 h | 24 h | 48 h | 4 d | 7 d |
|---|---|---|---|---|---|
| The experimental | 485 | 151 | 60 | 434 | 17 |
| group | 86 | 161 | 105 | 272 | 58 |
| | 177 | 170 | 50 | 142 | 13 |
| Mean value | 249 | 161 | 72 | 283 | 29 |
| The control | 389 | 328 | 303 | 1376 | 753 |
| group (Taxol) | 760 | 318 | 378 | 1023 | 550 |
| | 392 | 269 | 369 | 1574 | 145 |
| Mean value | 514 | 305 | 350 | 1324 | 483 |

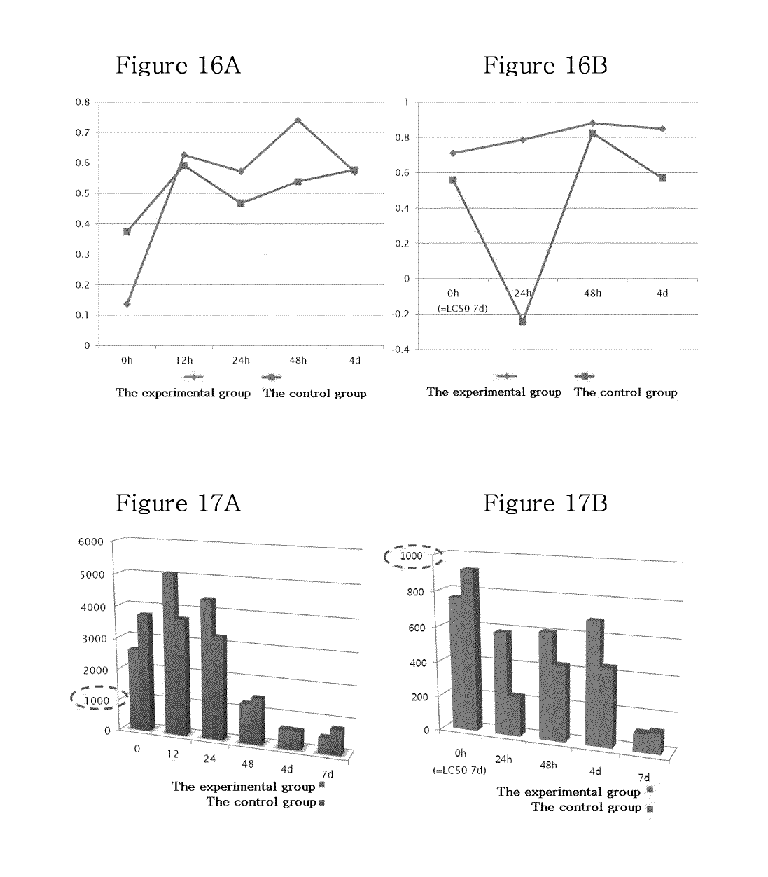

The following Table shows the absorbance, the viability and the cytotoxicity determined in cell line PC-3 during the 1^{st} week (LC50) (FIGS. 15 and 16).

TABLE 14

| | | | | | |
|---|---|---|---|---|---|
| Blank | 0 h | 12 h | 24 h | 48 h | 4 d |

The absorbance

| | | | | | | |
|---|---|---|---|---|---|---|
| The | 0.1469 | 1.0355 | 0.5369 | 0.6397 | 0.9032 | 1.4086 |
| experimental | 0.1564 | 0.8268 | 0.5526 | 0.7017 | 1.1264 | 1.0586 |
| group | 0.1597 | 0.8127 | 0.6271 | 0.6816 | 1.1159 | 1.3764 |
| Mean value | 0.1543 | 0.8917 | 0.5722 | 0.6743 | 1.0485 | 1.2812 |
| The control | 0.1574 | 0.6625 | 0.5779 | 0.7011 | 1.6838 | 1.0768 |
| group | 0.1598 | 0.7147 | 0.5126 | 0.7192 | 1.9128 | 1.1723 |
| (Taxol) | 0.1595 | 0.7027 | 0.6101 | 0.6542 | 1.6455 | 1.5491 |
| Mean value | 0.159 | 0.693 | 0.567 | 0.692 | 1.747 | 1.266 |
| The | 0.1587 | 0.8403 | 1.082 | 1.3307 | 3.468 | 2.7734 |
| untreated | 0.1544 | 0.868 | 1.1541 | 1.1019 | 3.4178 | 2.4704 |
| group | | | | | | |
| Mean value | 0.1566 | 0.8542 | 1.1181 | 1.2163 | 3.4429 | 2.6219 |
| The | | 0.7373 | 0.4179 | 0.5200 | 0.8942 | 1.1269 |
| experimental | | | | | | |
| group - | | | | | | |
| Blank | | | | | | |
| The control | | 0.5344 | 0.4080 | 0.5326 | 1.5885 | 1.1072 |
| group | | | | | | |
| (Taxol) - | | | | | | |
| Blank | | | | | | |

The viability

| | | | | | |
|---|---|---|---|---|---|
| The | 0.8632 | 0.3737 | 0.4275 | 0.2597 | 0.4298 |
| exper- | | | | | |
| imental | | | | | |
| group | | | | | |
| The | 0.6257 | 0.3649 | 0.4379 | 0.4614 | 0.4223 |
| control | | | | | |
| group | | | | | |
| (Taxol) | | | | | |

The cytotoxicity

| | | | | | |
|---|---|---|---|---|---|
| The | 0.1368 | 0.6263 | 0.5725 | 0.7403 | 0.5702 |
| exper- | | | | | |
| imental | | | | | |
| group | | | | | |
| The | 0.3743 | 0.5920 | 0.4674 | 0.5386 | 0.5777 |
| control | | | | | |
| group | | | | | |
| (Taxol) | | | | | |

The following Table shows the absorbance, the viability and the cytotoxicity determined in cell line PC-3 during the 2^{nd} week (recovery) (FIGS. 15 and 16).

TABLE 15

| | | | | |
|---|---|---|---|---|
| Blank | 0 h | 24 h | 48 h | 4 d |

The absorbance

| | | | | | |
|---|---|---|---|---|---|
| The | 0.1469 | 0.3907 | 0.4767 | 0.9343 | 0.5 |
| experimental | 0.1564 | 0.4147 | 0.374 | 0.5096 | 0.6055 |
| group | 0.1597 | 0.3994 | 0.3911 | 0.6206 | 2.5825 |
| Mean value | 0.1543 | 0.4016 | 0.4139 | 0.5651 | 0.5528 |
| The control | 0.1574 | 0.4893 | 1.9288 | 0.7081 | 1.072 |
| group (Taxol) | 0.1598 | 0.5937 | 1.3804 | 0.795 | 1.503 |
| | 0.1595 | 0.5232 | 1.6987 | 0.7948 | 2.9706 |
| Mean value | 0.1589 | 0.5354 | 1.6693 | 0.7660 | 1.2875 |
| The untreated | 0.1587 | 0.8403 | 1.3307 | 3.468 | 2.7734 |
| group | 0.1544 | 0.868 | 1.1019 | 3.4178 | 2.4704 |
| Mean value | 0.1566 | 0.8542 | 1.2163 | 3.4429 | 2.6219 |
| The | | 0.2473 | 0.2596 | 0.4108 | 0.3984 |
| experimental | | | | | |
| group - Blank | | | | | |
| The control | | 0.3765 | 1.5104 | 0.6071 | 1.1286 |
| group (Taxol) - | | | | | |
| Blank | | | | | |

The viability

| | | | | |
|---|---|---|---|---|
| The experimental | 0.2895 | 0.2134 | 0.1193 | 0.1520 |
| group | | | | |
| The control group | 0.4408 | 1.2418 | 0.1763 | 0.4305 |
| (Taxol) | | | | |

The cytotoxicity

| | | | | |
|---|---|---|---|---|
| The experimental | 0.7105 | 0.7866 | 0.8807 | 0.8480 |
| group | | | | |
| The control group | 0.5592 | −0.2418 | 0.8237 | 0.5695 |
| (Taxol) | | | | |

(6) HT 1299

HT 1299 is a non-small cell lung cancer. The cells have a homozygous partial deletion of the p53 protein, and lack expression of p53 protein. In this experiment, no cells appeared viable after treatment of the composition according to the invention.

The following Table shows the cell counts determined in cell line HT 1299 during the 1^{st} week (LC50) (FIG. 17).

TABLE 16

| | 0 h | 12 h | 24 h | 48 h | 4 d | 7 d |
|---|---|---|---|---|---|---|
| The | 2760 | 5750 | 4115 | 1250 | 655 | 539 |
| experimental | 3080 | 5620 | 4860 | 1574 | 685 | 496 |
| group | 1965 | 3845 | 4130 | 950 | 434 | 426 |
| Mean value | 2602 | 5072 | 4368 | 1258 | 591 | 487 |
| The control | 2270 | 3085 | 4255 | 1526 | 536 | 736 |
| group (Taxol) | 5520 | 5350 | 2690 | 1484 | 712 | 629 |
| | 3420 | 2680 | 2810 | 1382 | 488 | 982 |
| Mean value | 3737 | 3705 | 3252 | 1464 | 579 | 782 |
| The untreated | 2045 | 3825 | 4275 | 8818 | 9108 | 9131 |
| group | 2985 | 8630 | 7445 | 4061 | 8317 | 10597 |
| Mean value | 2515 | 6227.5 | 5860 | 6439.5 | 8712.5 | 9864 |

The following Table shows the cell counts determined in cell line HT 1299 during the 2^{nd} week (recovery) (FIG. 17).

TABLE 17

| | 0 h | 24 h | 48 h | 4 d | 7 d |
|---|---|---|---|---|---|
| The experimental | 331 | 766 | 747 | 704 | 68 |
| group | 1215 | 376 | 742 | 819 | 43 |
| | 740 | 612 | 339 | 549 | 202 |
| Mean value | 762 | 585 | 609 | 691 | 104 |
| The control | 789 | 129 | 499 | 545 | 144 |
| group (Taxol) | 776 | 262 | 386 | 470 | 107 |
| | 1193 | 291 | 410 | 322 | 104 |
| Mean value | 919 | 227 | 432 | 446 | 118 |

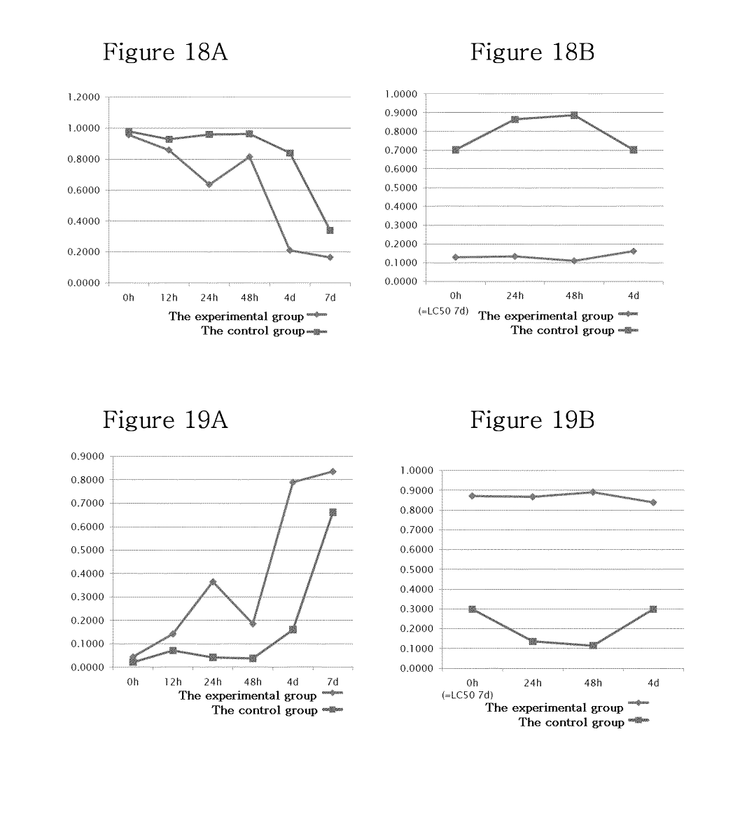

The following Table shows the absorbance, the viability and the cytotoxicity determined in cell line HT 1299 during the 1^{st} week (LC50) (FIGS. 18 and 19).

TABLE 18

| | | | | | | |
|---|---|---|---|---|---|---|
| Blank | 0 h | 12 h | 24 h | 48 h | 4 d | 7 d |

The absorbance

| | | | | | | | |
|---|---|---|---|---|---|---|---|
| The | 0.2039 | 2.5484 | 2.6886 | 2.1475 | 1.8847 | 0.7293 | 0.6839 |
| experimental | 0.2139 | 2.5409 | 2.6915 | 0.9981 | 2.4107 | 0.8923 | 0.7057 |
| group | 0.2351 | 2.9103 | 2.6957 | 2.6847 | 2.4211 | 0.8973 | 0.8328 |
| Mean value | 0.2176 | 2.6665 | 2.6919 | 1.9434 | 2.2388 | 0.8396 | 0.7408 |
| The control | 0.2078 | 2.6703 | 2.8787 | 2.8128 | 2.6333 | 2.5786 | 1.504 |
| group (Taxol) | 0.212 | 2.7643 | 2.8939 | 2.7759 | 2.5408 | 2.817 | 1.2209 |
| | 0.2119 | 2.716 | 2.8933 | 2.8539 | 2.6225 | 2.6791 | 1.1381 |
| Mean value | 0.2106 | 2.7169 | 2.8886 | 2.8142 | 2.5989 | 2.6916 | 1.2877 |
| The untreated | 0.2188 | 2.5719 | 2.9371 | 2.679 | 2.5226 | 2.8134 | 3.2258 |
| group | 0.2185 | 2.5463 | 2.8277 | 2.7505 | 2.4367 | 3.0896 | 3.131 |
| Mean value | 0.2187 | 2.5591 | 2.8824 | 2.7148 | 2.4797 | 2.9515 | 3.1784 |
| The | | 2.4489 | 2.4743 | 1.7258 | 2.0212 | 0.6220 | 0.5232 |
| experimental | | | | | | | |
| group - Blank | | | | | | | |
| The control | | 2.5063 | 2.6781 | 2.6036 | 2.3883 | 2.4810 | 1.0771 |
| group (Taxol) - | | | | | | | |
| Blank | | | | | | | |

The viability

| | | | | | | |
|---|---|---|---|---|---|---|
| The | 0.9569 | 0.8584 | 0.6357 | 0.8151 | 0.2107 | 0.1646 |
| experimental | | | | | | |
| group | | | | | | |
| The control | 0.9794 | 0.9291 | 0.9591 | 0.9632 | 0.8406 | 0.3389 |
| group (Taxol) | | | | | | |

The cytotoxicity

| | | | | | | |
|---|---|---|---|---|---|---|
| The | 0.0431 | 0.1416 | 0.3643 | 0.1849 | 0.7893 | 0.8354 |
| experimental | | | | | | |
| group | | | | | | |
| The control | 0.0206 | 0.0709 | 0.0409 | 0.0368 | 0.1594 | 0.6611 |
| group (Taxol) | | | | | | |

The following Table shows the absorbance, the viability and the cytotoxicity determined in cell line HT 1299 the 2^{nd} week (recovery) (FIGS. 18 and 19).

TABLE 19

| | | | | |
|---|---|---|---|---|
| Blank | 0 h | 24 h | 48 h | 4 d |

The absorbance

| | | | | | |
|---|---|---|---|---|---|
| The | 0.2832 | 0.5159 | 0.5905 | 0.5858 | 0.7835 |
| experimental | 0.2435 | 0.5922 | 0.6428 | 0.4964 | 0.6774 |
| group | 0.267 | 0.6731 | 0.6448 | 0.5245 | 0.7615 |
| Mean value | 0.2646 | 0.5937 | 0.6260 | 0.5356 | 0.7408 |
| The control | 0.2567 | 2.3962 | 2.6093 | 2.9676 | 2.4054 |
| group | 0.2744 | 1.9675 | 2.6918 | 3.2735 | 2.6902 |
| (Taxol) | 0.2857 | 1.8392 | 2.5521 | 1.1675 | 1.9256 |
| Mean value | 0.2723 | 2.0676 | 2.6177 | 2.4695 | 2.3404 |
| The untreated | | | | | |
| group | | | | | |
| Mean value | 0.2187 | 2.5591 | 2.7148 | 2.4797 | 2.9515 |
| The | | 0.3292 | 0.3615 | 0.2710 | 0.4762 |
| experimental | | | | | |
| group - Blank | | | | | |
| The control | | 1.7954 | 2.3455 | 2.1973 | 2.0681 |
| group | | | | | |
| (Taxol) - Blank | | | | | |

The viability

| | | | | |
|---|---|---|---|---|
| The experimental | 0.1286 | 0.1331 | 0.1093 | 0.1614 |
| group | | | | |
| The control | 0.7016 | 0.8640 | 0.8861 | 0.7007 |
| group (Taxol) | | | | |

The cytotoxicity

| | | | | |
|---|---|---|---|---|
| The experimental | 0.8714 | 0.8669 | 0.8907 | 0.8386 |
| group | | | | |
| The control | 0.2984 | 0.1360 | 0.1139 | 0.2993 |
| group (Taxol) | | | | |

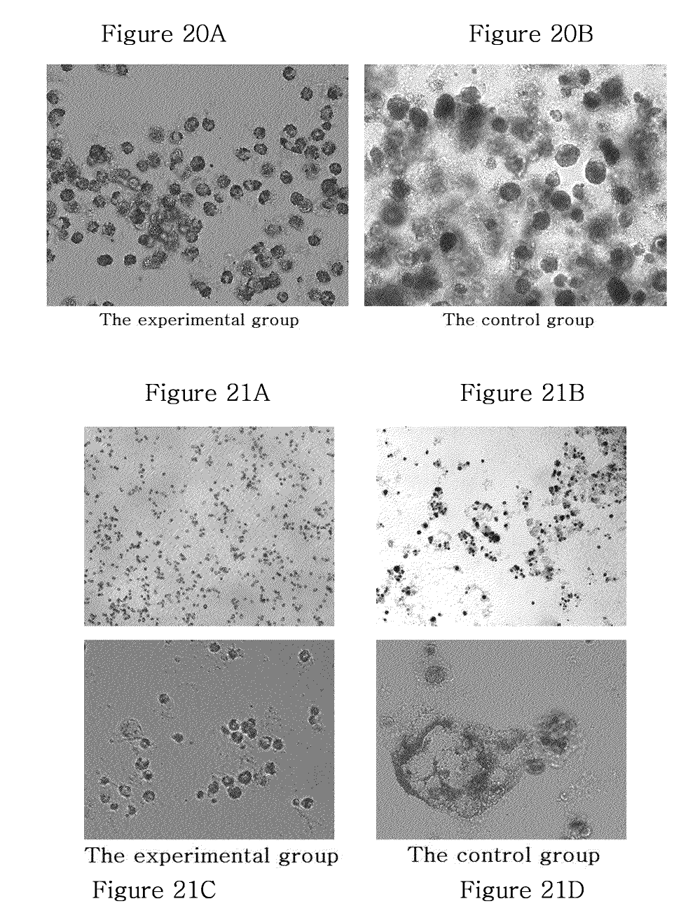

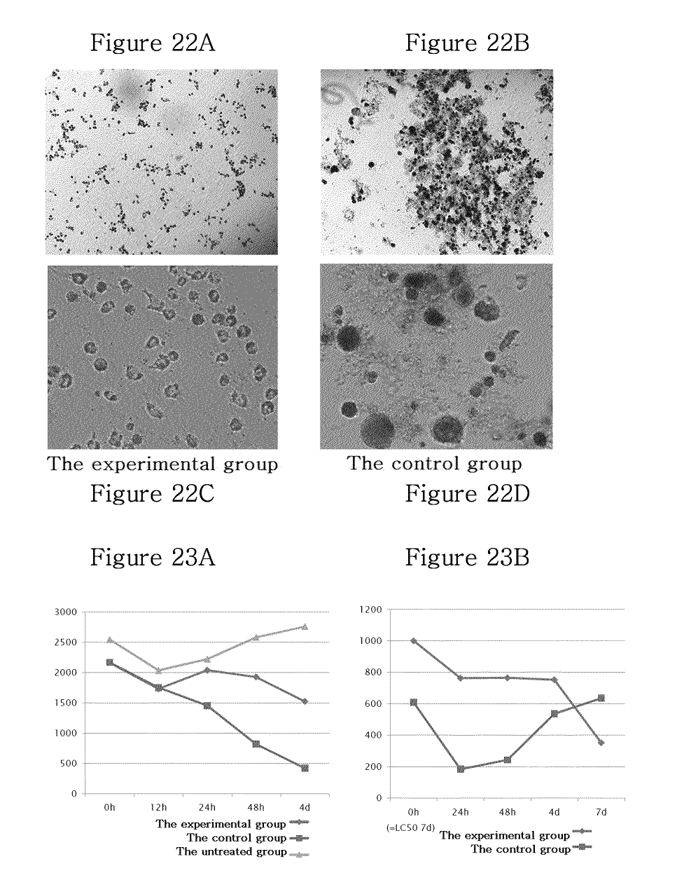

In addition, the control group showed that cell membranes were ruptured by toxicity of Taxol, and then nuclei of cells changed into black. This is similar to a general process that cells die. However, the experimental group showed a unique and interesting phenomenon wherein nuclei of the cells first burst with cell membranes changing into black, and then cell membranes were ruptured (FIGS. 20 to 22).

(7) Saos-2

Saos-2 is bone cancer cell line.

The following Table shows the cell counts determined in cell line Saos-2 during the 1^{st} week (LC50) (FIG. 23).

TABLE 20

| | 0 h | 12 h | 24 h | 48 h | 4 d |
|---|---|---|---|---|---|
| The experimental | 2016 | 1647 | 2040 | 1982 | 1494 |
| group | 2151 | 1804 | 1978 | 1940 | 1652 |
| | 2314 | 1735 | 2094 | 1852 | 1420 |
| Mean value | 2160 | 1729 | 2037 | 1925 | 1522 |
| The control | 2221 | 1790 | 1376 | 822 | 570 |
| group (Taxol) | 2135 | 1705 | 1452 | 776 | 435 |
| | 2143 | 1756 | 1529 | 857 | 255 |
| Mean value | 2166 | 1750 | 1452 | 818 | 420 |
| The untreated | 2726 | 1740 | 2234 | 2406 | 4421 |
| group | 2361 | 2325 | 2203 | 2755 | 1034 |
| Mean value | 2544 | 2033 | 2219 | 2581 | 2728 |

The following Table shows the cell counts determined in cell line Saos-2 during the 2^{nd} week (recovery) (FIG. 23).

TABLE 21

| | 0 h | 24 h | 48 h | 4 d | 7 d |
|---|---|---|---|---|---|
| The experimental | 995 | 722 | 707 | 912 | 263 |
| group | 1026 | 800 | 787 | 670 | 454 |
| | 980 | 764 | 798 | 674 | 337 |
| Mean value | 1000 | 762 | 764 | 752 | 351 |
| The control | 291 | 165 | 249 | 589 | 289 |
| group (Taxol) | 759 | 126 | 165 | 602 | 793 |
| | 778 | 258 | 312 | 416 | 822 |
| Mean value | 609 | 183 | 242 | 536 | 635 |

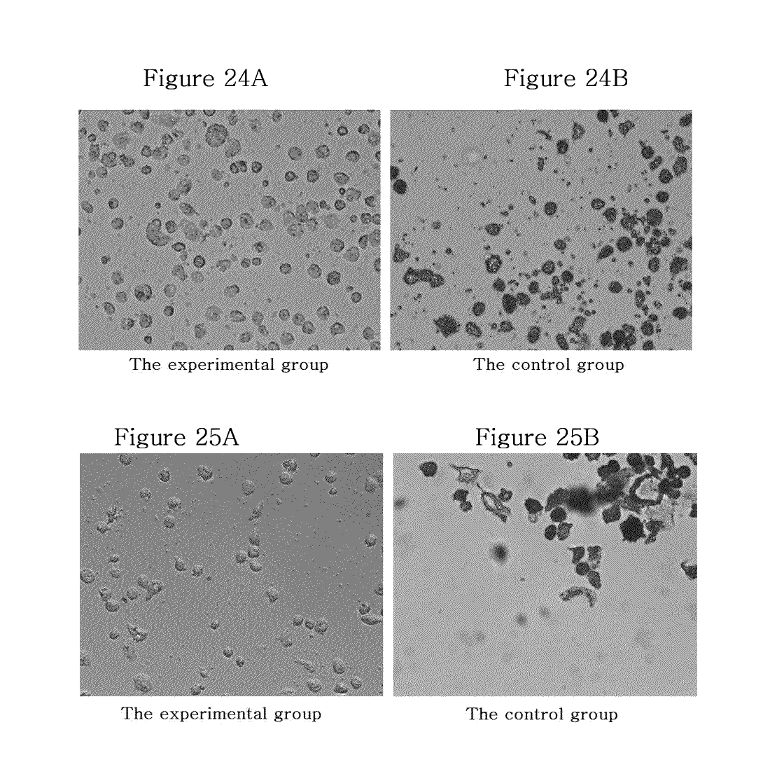

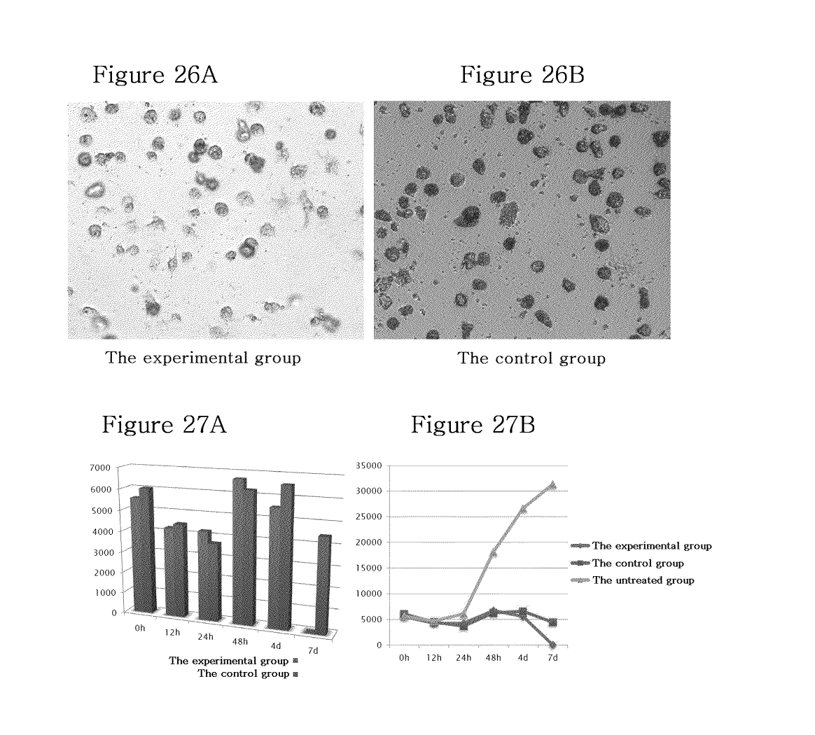

In addition, the control group showed that cell membranes were ruptured by toxicity of Taxol, and then nuclei of cells changed into black. This is similar to a general process that cells die. However, the experimental group showed a unique and interesting phenomenon wherein nuclei of the cells first burst with cell membranes changing into black, and then cell membranes were ruptured (FIGS. 24 to 26).

(8) C-6

C-6 is glioblastoma cells. Glioblastoma multiforme (GBM) is the most common malignant form of glioma, and resistant to therapeutic interventions, causing most patients to die within 1 year after diagnosis. The rat C-6 gliomacell line, originally produced by Wistar-Furth rats exposed to N,N′-nitroso-methylurea, is morphologically similar to GBM when injected into brain of rats and has been used as both in vivo and in vitro model for the study of this kind of tumor.

During the experiment, it proved resistant to Taxol. Whereas, after 4 days of exposure to the composition of the invention, the cells were killed and did not recover.

The following Table shows the cell counts determined in cell line C-6 during the 1^{st} week (LC50) (FIG. 27).

TABLE 22

| | 0 h | 12 h | 24 h | 48 h | 4 d | 7 d |
|---|---|---|---|---|---|---|
| The | 5035 | 4600 | 2464 | 7947 | 7116 | 17 |
| experimental | 6102 | 3934 | 4777 | 6888 | 3248 | 8 |
| group | 5525 | 4234 | 5447 | 5283 | 6303 | 30 |
| Mean value | 5554 | 4256 | 4229 | 6706 | 5556 | 18 |
| The control | 7077 | 5442 | 3853 | 5378 | 6751 | 2671 |
| group (Taxol) | 4897 | 4240 | 3340 | 6722 | 6905 | 3426 |
| | 6111 | 3726 | 3895 | 6630 | 6026 | 7253 |
| Mean value | 6028 | 4469 | 3696 | 6243 | 6561 | 4450 |
| The untreated | 5384 | 5320 | 5877 | 20286 | 29190 | 32825 |
| group | 5873 | 4002 | 6305 | 15960 | 24075 | 29837 |
| Mean value | 5629 | 4661 | 6091 | 18123 | 26633 | 31331 |

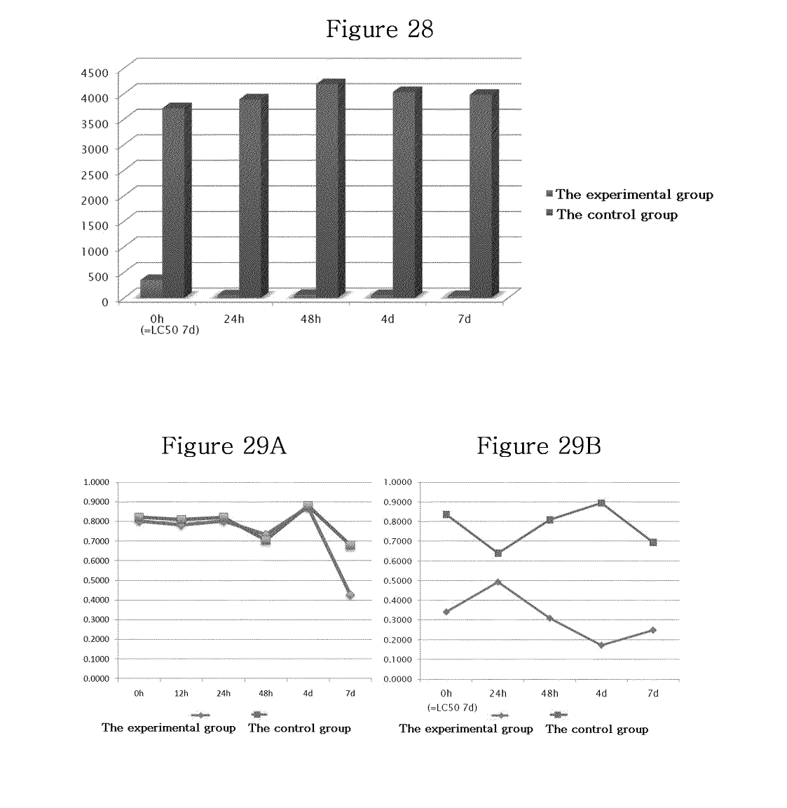

The following Table shows the cell counts determined in cell line C-6 during the 2^{nd} week (recovery) (FIG. 28).

TABLE 23

| | 0 h | 24 h | 48 h | 4 d | 7 d |
|---|---|---|---|---|---|
| The experimental | 542 | 54 | 105 | 38 | 34 |
| group | 231 | 39 | 33 | 95 | 31 |
| | 271 | 58 | 39 | 27 | 43 |
| Mean value | 348 | 50 | 59 | 53 | 36 |
| The control | 3948 | 4285 | 5457 | 4083 | 4316 |
| group (Taxol) | 4207 | 3984 | 3930 | 3184 | 3634 |
| | 2984 | 3392 | 3166 | 4825 | 3989 |
| Mean value | 3713 | 3887 | 4184 | 4031 | 3980 |

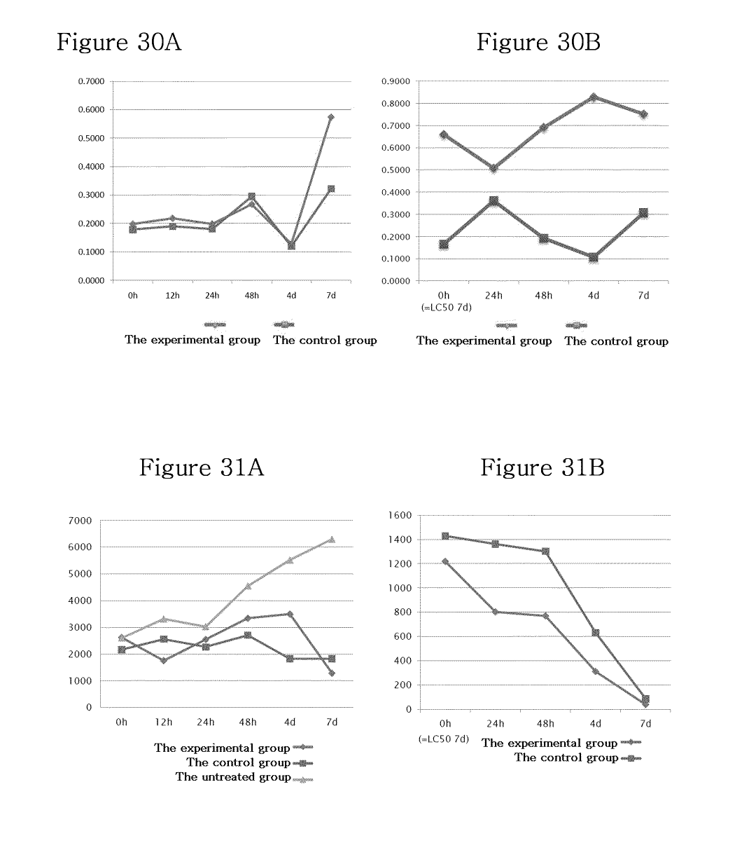

The following Table shows the absorbance, the viability and the cytotoxicity determined in cell line C-6 during the 1^{st} week (LC50) (FIGS. 29 and 30).

TABLE 24

| | | | | | | |
|---|---|---|---|---|---|---|
| Blank | 0 h | 12 h | 24 h | 48 h | 4 d | 7 d |

The absorbance

| | | | | | | | |
|---|---|---|---|---|---|---|---|
| The | 0.2784 | 2.4419 | 2.7 | 2.4316 | 1.9171 | 2.0187 | 1.5397 |
| experimental | 0.499 | 2.3204 | 2.3552 | 2.5843 | 2.1239 | 2.251 | 1.8557 |
| group | 0.5452 | 2.5977 | 2.8205 | 2.5768 | 3.3616 | 3.6078 | 1.3283 |
| Mean value | 0.4409 | 2.4533 | 2.6252 | 2.5309 | 2.4675 | 2.6258 | 1.5746 |
| The control | 0.5034 | 2.482 | 2.6595 | 2.7106 | 2.5122 | 2.3613 | 2.3017 |
| group (Taxol) | 0.479 | 2.6477 | 2.851 | 2.6263 | 2.5907 | 3.4031 | 2.334 |
| | 0.5846 | 2.6197 | 2.8475 | 2.6408 | 2.3173 | 2.4181 | 2.3441 |
| Mean value | 0.5223 | 2.5831 | 2.7860 | 2.6592 | 2.4734 | 2.7275 | 2.3266 |
| The untreated | 0.4159 | 2.568 | 2.7277 | 2.6513 | 2.7968 | 2.6523 | 2.6488 |
| group | 0.2362 | 2.4479 | 2.8599 | 2.5606 | 2.7347 | 2.3571 | 2.6727 |
| Mean value | 0.3261 | 2.5080 | 2.7938 | 2.6060 | 2.7658 | 2.5047 | 2.6608 |
| The | | 2.0125 | 2.1844 | 2.0900 | 2.0267 | 2.1850 | 1.1337 |
| experimental | | | | | | | |
| group - Blank | | | | | | | |
| The control | | 2.0608 | 2.2637 | 2.1369 | 1.9511 | 2.2052 | 1.8043 |
| group (Taxol) - | | | | | | | |
| Blank | | | | | | | |

The viability

| | | | | | | |
|---|---|---|---|---|---|---|
| The | 0.8024 | 0.7819 | 0.8020 | 0.7328 | 0.8723 | 0.4261 |
| experimental | | | | | | |
| group | | | | | | |
| The control | 0.8217 | 0.8102 | 0.8200 | 0.7054 | 0.8804 | 0.6781 |
| group (Taxol) | | | | | | |

The cytotoxicity

| | | | | | | |
|---|---|---|---|---|---|---|
| The | 0.1976 | 0.2181 | 0.1980 | 0.2672 | 0.1277 | 0.5739 |
| experimental | | | | | | |
| group | | | | | | |
| The control | 0.1783 | 0.1898 | 0.1800 | 0.2946 | 0.1196 | 0.3219 |
| group (Taxol) | | | | | | |

The following Table shows the absorbance, the viability and the cytotoxicity determined in cell line C-6 during the 2^{nd} week (recovery) (FIGS. 29 and 30).

TABLE 25

| | | | | | |
|---|---|---|---|---|---|
| Blank | 0 h | 24 h | 48 h | 4 d | 7 d |

The absorbance

| | | | | | | |
|---|---|---|---|---|---|---|
| The | 0.2784 | 0.969 | 1.77 | 1.5914 | 0.9087 | 1.2024 |
| experimental | 0.499 | 1.5505 | 1.5576 | 1.0755 | 0.5892 | 0.9067 |
| group | 0.5452 | 1.3674 | 1.8463 | 1.2193 | 1.1168 | 1.1978 |
| Mean value | 0.4409 | 1.2956 | 1.7246 | 1.2954 | 0.8716 | 1.1023 |
| The control | 0.5034 | 2.6926 | 2.2634 | 2.7053 | 2.8075 | 2.4909 |
| group | 0.479 | 2.7171 | 1.9908 | 3.111 | 2.6888 | 2.4639 |
| (Taxol) | 0.5846 | 2.4409 | 2.3106 | 2.4572 | 2.7862 | 2.1497 |
| Mean value | 0.5223 | 2.6169 | 2.1883 | 2.7578 | 2.7608 | 2.3682 |
| The | 0.4159 | 2.568 | 2.6513 | 2.7968 | 2.6523 | 2.6488 |
| untreated | 0.2362 | 2.4479 | 2.5606 | 2.7347 | 2.3571 | 2.6727 |
| group | | | | | | |
| Mean value | 0.3261 | 2.5080 | 2.6060 | 2.7658 | 2.5047 | 2.6608 |
| The | | 0.8548 | 1.2838 | 0.8545 | 0.4307 | 0.6614 |
| experimental | | | | | | |
| group - | | | | | | |
| Blank | | | | | | |
| The control | | 2.0945 | 1.6659 | 2.2355 | 2.2385 | 1.8458 |
| group | | | | | | |
| (Taxol) - | | | | | | |
| Blank | | | | | | |

The viability

| | | | | | |
|---|---|---|---|---|---|
| The | 0.3408 | 0.4926 | 0.3090 | 0.1720 | 0.249 |
| exper- | | | | | |
| imental | | | | | |
| group | | | | | |
| The | 0.8352 | 0.6393 | 0.8083 | 0.8937 | 0.694 |
| control | | | | | |
| group | | | | | |
| (Taxol) | | | | | |

The cytotoxicity

| | | | | | |
|---|---|---|---|---|---|
| The | 0.6592 | 0.5074 | 0.6910 | 0.8280 | 0.7514 |
| exper- | | | | | |
| imental | | | | | |
| group | | | | | |
| The | 0.1648 | 0.3607 | 0.1917 | 0.1063 | 0.3063 |
| control | | | | | |
| group | | | | | |
| (Taxol) | | | | | |

(9) AsPc

AsPc is a pancreatic cancer cell line.

The following Table shows the cell counts determined in cell line AsPc during the 1^{st} week (LC50) (FIG. 31).

TABLE 26

| | 0 h | 12 h | 24 h | 48 h | 4 d | 7 d |
|---|---|---|---|---|---|---|
| The | 2743 | 1602 | 2705 | 2420 | 1640 | 1350 |
| experimental | 2601 | 1762 | 2454 | 1920 | 1480 | 1710 |
| group | 2519 | 1898 | 2480 | 2177 | 1847 | 790 |
| Mean value | 2621 | 1754 | 2546 | 2172 | 1656 | 1283 |
| The control | 2039 | 2279 | 2161 | 2130 | 2020 | 2174 |
| group (Taxol) | 2111 | 2550 | 2310 | 1800 | 2100 | 1902 |
| | 2340 | 2833 | 2336 | 2810 | 2007 | 1393 |
| Mean value | 2163 | 2554 | 2269 | 2247 | 2042 | 1823 |
| The untreated | 2796 | 2705 | 2941 | 4416 | 5693 | 7700 |
| group | 2402 | 3917 | 3106 | 4682 | 5343 | 4900 |
| Mean value | 2599 | 3311 | 3023.5 | 4549 | 5518 | 6300 |

The following Table shows the cell counts determined in cell line AsPc during the 2^{nd} week (recovery) (FIG. 31).

TABLE 27

| | 0 h | 24 h | 48 h | 4 d | 7 d |
|---|---|---|---|---|---|
| The experimental | 1180 | 1242 | 1135 | 475 | 82 |
| group | 1281 | 1164 | 1171 | 457 | 32 |
| | 1192 | 1148 | 790 | 470 | 42 |
| Mean value | 1218 | 802 | 769 | 311 | 38 |
| The control | 1213 | 1372 | 1490 | 327 | 101 |
| group (Taxol) | 1770 | 1357 | 1202 | 724 | 85 |
| | 1301 | 1358 | 1210 | 842 | 73 |
| Mean value | 1428 | 1362 | 1301 | 631 | 86 |

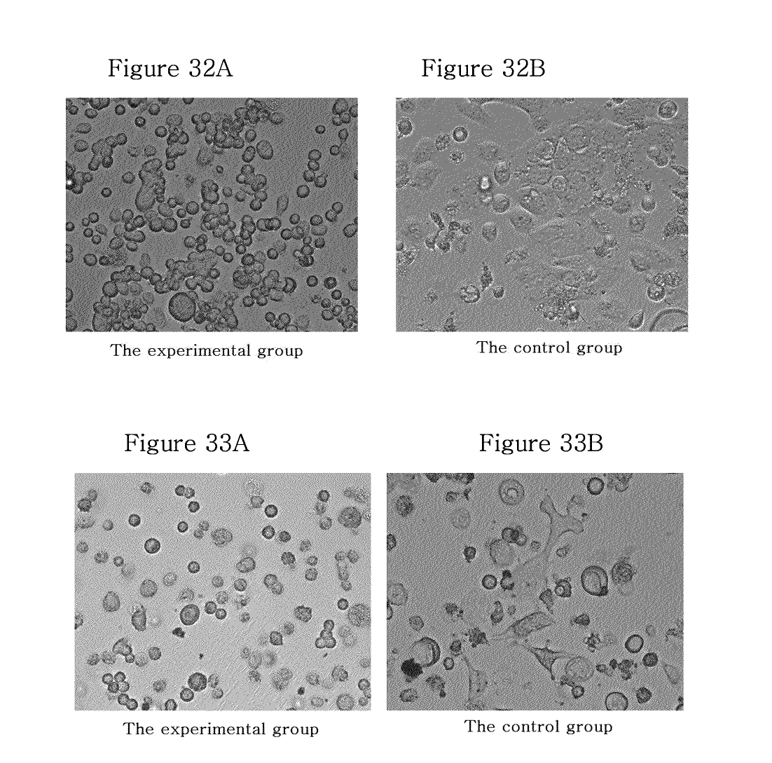

It was observed that membranes of cancer cells changed into black and were dying in the experimental group. In such a case, there is very little possibility that cancer cells will regenerate (FIG. 32).

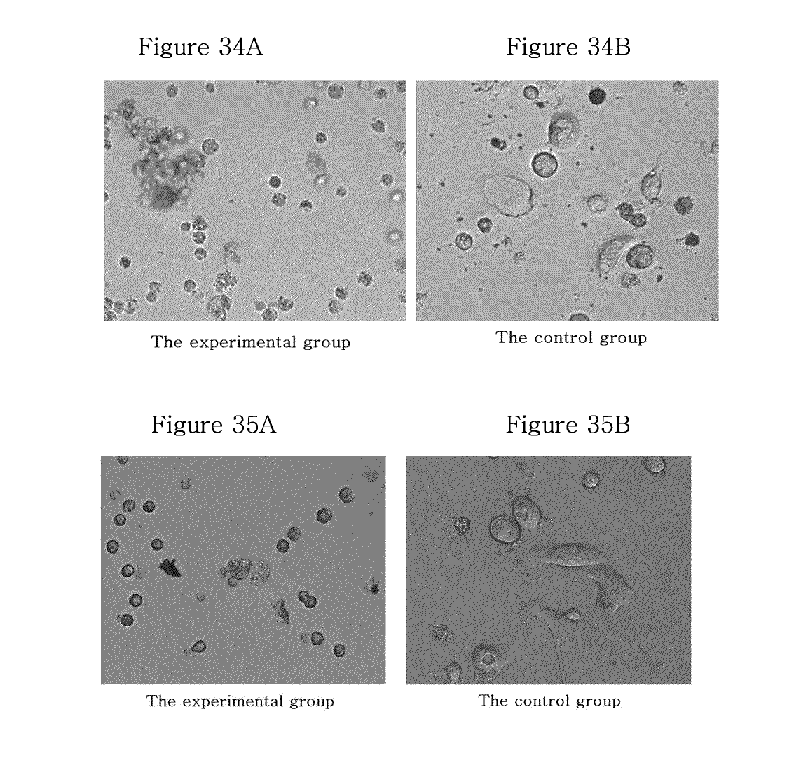

In addition, the control group showed a general process of cell death that cell membranes were ruptured by toxicity of Taxol, and then nuclei of cells changed into black. However, the experimental group showed a unique and interesting phenomenon wherein nuclei of the cells first burst with cell membranes changing into black, and then cell membranes were ruptured (FIGS. 33 to 35).

(10) HME 50 HT—Normal Epithelial Cells

Human Mammary Epithelial (HME) cells were derived from adjacent normal tissue.

During the experiment, no cytotoxicity was observed in the group treated with the composition according to the invention. Surprisingly, the HME 50 HT cells thrived and became over confluent during recovery. And, it was found from the photomicrographs that the cells appeared very healthy and unstressed.

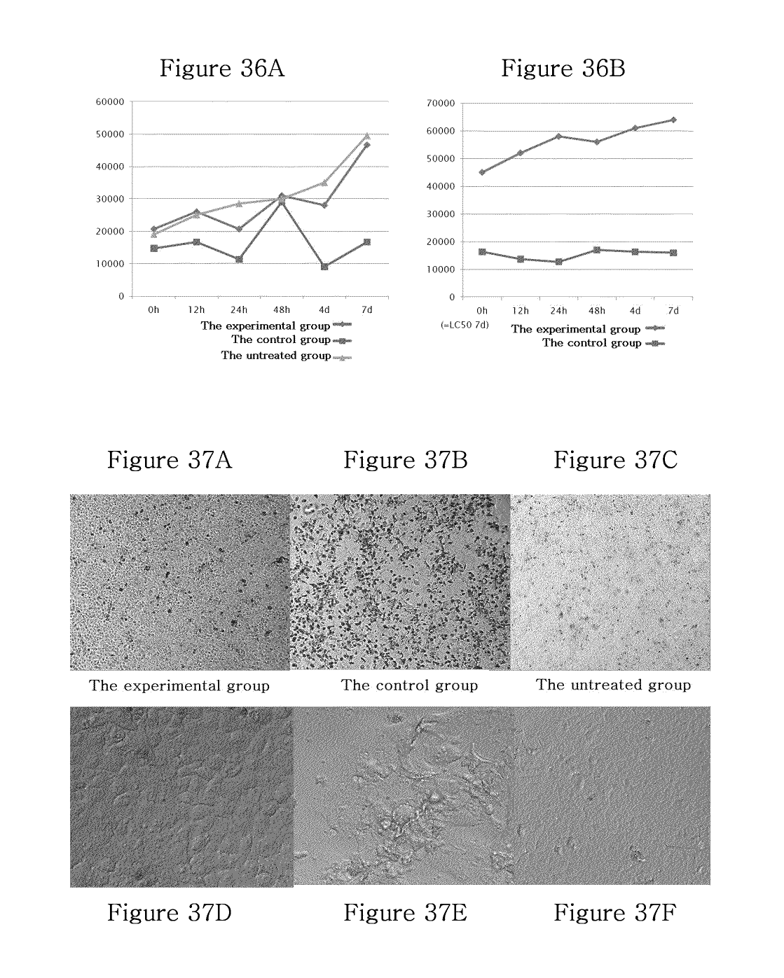

The following Table shows the cell counts determined in cell line HME during the 1^{st} week (LC50) (FIG. 36).

TABLE 28

| | 0 h | 12 h | 24 h | 48 h | 4 d | 7 d |
|---|---|---|---|---|---|---|
| The | 17000 | 25000 | 20000 | 26000 | 28000 | 41000 |
| experimental | 22000 | 26000 | 21000 | 28000 | 28000 | 49000 |
| group | 23000 | 27000 | 21000 | 25000 | 28000 | 66000 |
| Mean value | 20667 | 26000 | 20667 | 26333 | 28000 | 46667 |
| The control | 11000 | 23000 | 11000 | 18000 | 9000 | 19000 |
| group (Taxol) | 21000 | 13000 | 9000 | 11000 | 9000 | 14000 |
| | 12000 | 14000 | 14000 | 18000 | 9000 | 17000 |
| Mean value | 14667 | 16667 | 11333 | 15667 | 9000 | 16667 |
| The untreated | 18000 | 25000 | 28000 | 31000 | 32000 | 67000 |
| group | 20000 | 25000 | 29000 | 29000 | 38000 | 32000 |
| Mean value | 19000 | 25000 | 28500 | 30000 | 35000 | 49500 |

The following Table shows the cell counts determined in cell line HME during the 2^{nd} week (recovery) (FIG. 36).

TABLE 29

| | 0 h | 12 h | 24 h | 48 h | 4 d | 7 d |
|---|---|---|---|---|---|---|
| The | 50000 | 52000 | 60000 | 56000 | 60000 | 59000 |
| experimental | 46000 | 57000 | 52000 | 57000 | 58000 | 69000 |
| group | 39000 | 47000 | 62000 | 55000 | 65000 | 64000 |
| Mean value | 45000 | 52000 | 58000 | 56000 | 61000 | 64000 |
| The control | 17000 | 13000 | 15000 | 16000 | 19000 | 19000 |
| group (Taxol) | 13000 | 15000 | 11000 | 17000 | 12000 | 15000 |
| | 19000 | 13000 | 12000 | 18000 | 18000 | 14000 |
| Mean value | 16333 | 13667 | 12667 | 17000 | 16333 | 16000 |

In the experimental group, it was observed that normal cells remained very healthy, and successive cell division and cell growth appeared. Furthermore, though normal cells treated with the composition of the invention were exposed for one week, the number of cells increased during both the 1^{st} week (LC50) and the 2^{nd} week (recovery). Such cell growth in the experimental group is similar to that in the untreated group, and this means the composition according to the invention does not negatively affect normal cells.

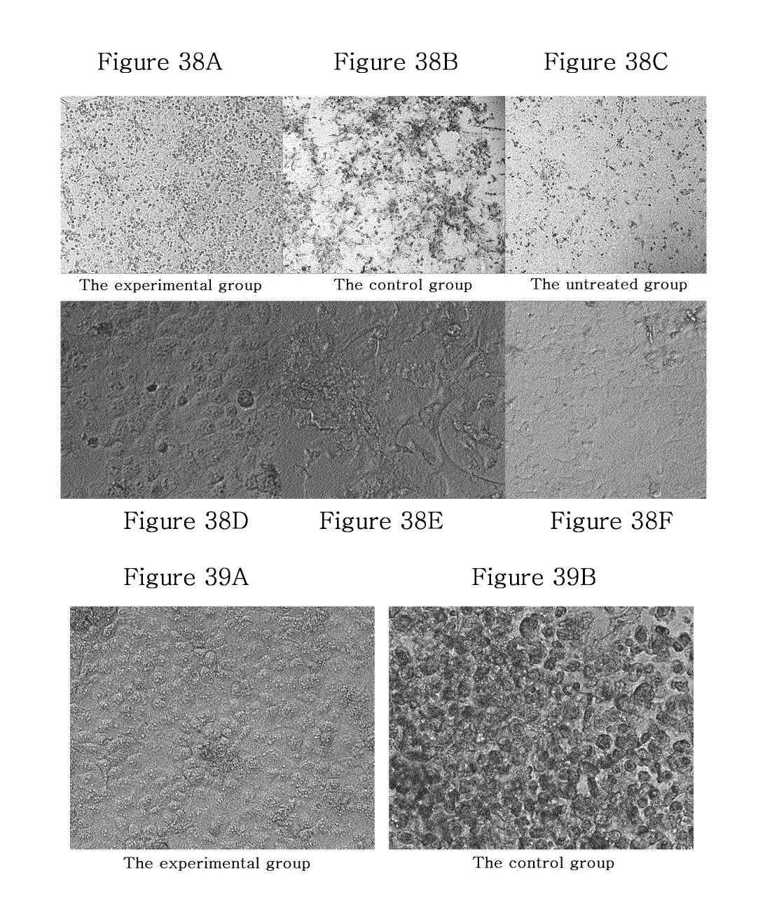

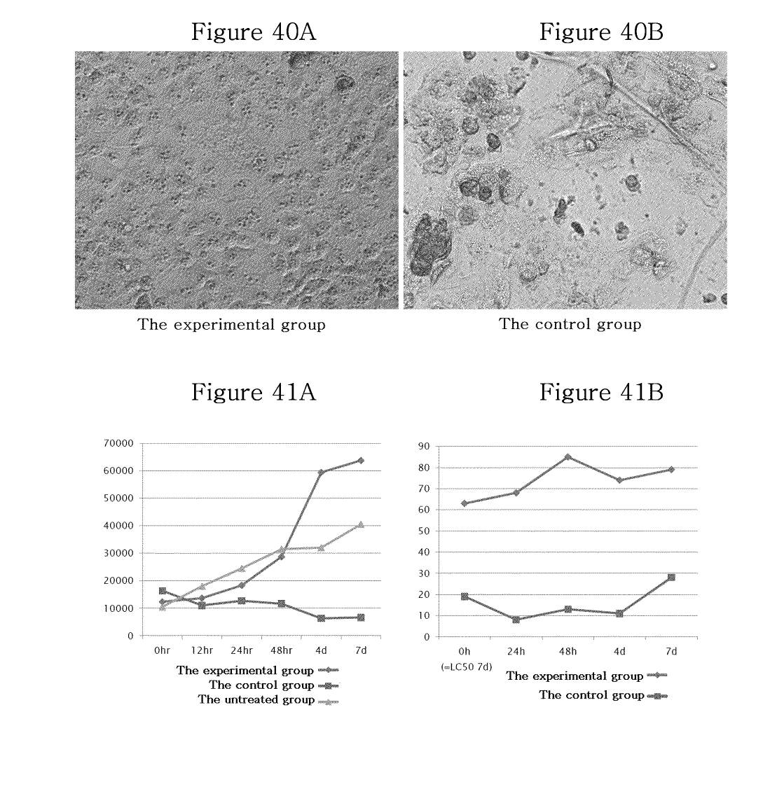

In contrast, the group treated with Taxol showed that the number of cells did not increase, and was merely maintained for two weeks despite exposure for only 24 hours. And, it was observed in the control group that normal cells were destroyed and suffered a great deal of strain. This means normal cells were under a lot of stress by Taxol (FIGS. 37 to 40).

(11) BJ—Normal Fibroblasts

BJ cells were derived from normal foreskin of a newborn, and have a long lifespan in comparison with other normal human fibroblast cells. Although they have the capacity to proliferate to a maximum of 72 population doublings before the onset of senescence, they are telomerase negative.

The group treated with the composition according to the invention showed no toxicity, and the cells were very healthy and unstressed. Also, enhanced cell growth was observed.

The following Table shows the cell counts determined in cell line BJ during the 1^{st} week (LC50) (FIG. 41).

TABLE 30

| | 0 h | 12 h | 24 h | 48 h | 4 d | 7 d |
|---|---|---|---|---|---|---|
| The | 13000 | 14000 | 20000 | 24000 | 59000 | 63000 |
| experimental | 9000 | 10000 | 22000 | 34000 | 61000 | 67000 |
| group | 15000 | 17000 | 13000 | 28000 | 58000 | 61000 |
| Mean value | 12333 | 13667 | 18333 | 28667 | 59333 | 63667 |
| The control | 17000 | 9000 | 17000 | 10000 | 5000 | 9000 |
| group (Taxol) | 18000 | 12000 | 11000 | 11000 | 5000 | 4000 |
| | 14000 | 12000 | 10000 | 14000 | 9000 | 7000 |
| Mean value | 16333 | 11000 | 12667 | 11667 | 6333 | 6667 |
| The untreated | 10000 | 20000 | 24000 | 29000 | 33000 | 47000 |
| group | 11000 | 16000 | 25000 | 34000 | 31000 | 34000 |
| Mean value | 10500 | 18000 | 24500 | 31500 | 32000 | 40500 |

The following Table shows the cell counts determined in cell line BJ during the 2^{nd} week (recovery) (FIG. 41).

TABLE 31

| | 0 h | 24 h | 48 h | 4 d | 7 d |
|---|---|---|---|---|---|
| The | 56 | 67 | 100 | 77 | 63 |
| experimental | 73 | 72 | 74 | 88 | 85 |
| group | 60 | 65 | 82 | 56 | 90 |
| Mean value | 63 | 68 | 85 | 74 | 79 |
| The control | 49 | 7 | 14 | 7 | 29 |
| group (Taxol) | 4 | 9 | 16 | 18 | 19 |
| | 5 | 7 | 8 | 8 | 35 |
| Mean value | 19 | 8 | 13 | 11 | 28 |

It was observed that normal cells in the experimental group proliferated more than in the untreated group, whereas normal cells in the control group decreased due to stress by toxicity of Taxol, and recorved after 4 days of the 2^{nd} week (recovery).

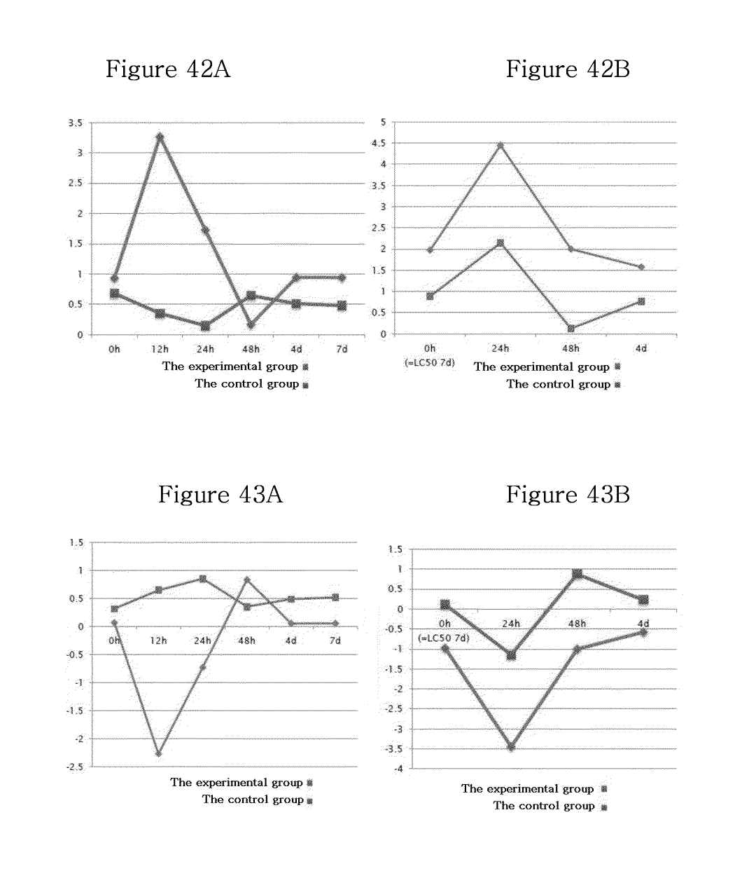

The following Table shows the absorbance, the viability and the cytotoxicity determined in cell line BJ during the 1^{st} week (LC50) (FIGS. 42 and 43).

TABLE 32

| | | | | | | |
|---|---|---|---|---|---|---|
| Blank | 0 h | 12 h | 24 h | 48 h | 4 d | 7 d |

The absorbance

| | | | | | | | |
|---|---|---|---|---|---|---|---|
| The | 0.2397 | 1.5202 | 1.9785 | 2.2981 | 0.5769 | 2.3792 | 2.1166 |
| experimental | 0.2345 | 1.4973 | 1.8766 | 2.1675 | 0.5885 | 2.4592 | 2.6472 |
| group | 0.2426 | 1.4718 | 1.837 | 2.1045 | 0.5601 | 2.2919 | 2.1845 |
| Mean value | 0.2389 | 1.4964 | 1.8974 | 2.1900 | 0.5752 | 2.3768 | 2.3161 |
| The control | 0.3226 | 1.2268 | 0.5027 | 0.4879 | 1.6842 | 1.4766 | 1.4436 |
| group (Taxol) | 0.3239 | 1.2557 | 0.504 | 0.492 | 1.6877 | 1.4687 | 1.3256 |
| | 0.3176 | 1.2397 | 0.4951 | 0.4863 | 1.5017 | 1.4879 | 1.3677 |
| Mean value | 0.3214 | 1.2407 | 0.5006 | 0.4887 | 1.6245 | 1.4777 | 1.3790 |
| The untreated | 0.3183 | 1.3311 | 0.5024 | 0.5019 | 2.0492 | 2.1943 | 2.1956 |
| group | 0.3231 | 1.3661 | 0.5128 | 1.7542 | 1.987 | 2.325 | 2.203 |
| Mean value | 0.3207 | 1.3486 | 0.5076 | 1.1281 | 2.0181 | 2.2597 | 2.1993 |
| The | | 1.2575 | 1.6584 | 1.9511 | 0.3362 | 2.1378 | 2.0772 |
| experimental | | | | | | | |
| group - Blank | | | | | | | |
| The control | | 0.9194 | 0.1792 | 0.1674 | 1.3032 | 1.1564 | 1.0576 |
| group (Taxol) - | | | | | | | |
| Blank | | | | | | | |

The viability

| | | | | | | |
|---|---|---|---|---|---|---|
| The | 0.9324 | 3.2672 | 1.7296 | 0.1666 | 0.9461 | 0.9445 |
| experimental | | | | | | |
| group | | | | | | |
| The control | 0.6817 | 0.3531 | 0.1484 | 0.6457 | 0.5117 | 0.4809 |
| group (Taxol) | | | | | | |

The cytotoxicity

| | | | | | | |
|---|---|---|---|---|---|---|
| The | 0.0676 | −2.2672 | −0.7296 | 0.8334 | 0.0539 | 0.0555 |
| experimental | | | | | | |
| group | | | | | | |
| The control | 0.3183 | 0.6469 | 0.8516 | 0.3543 | 0.4883 | 0.5191 |
| group (Taxol) | | | | | | |

The following Table shows the absorbance, the viability and the cytotoxicity determined in cell line BJ during the 2^{nd} week (recovery) (FIGS. 42 and 43).

TABLE 33

| | | | | |
|---|---|---|---|---|
| Blank | 0 h | 24 h | 48 h | 4 d |

The absorbance

| | | | | | |
|---|---|---|---|---|---|
| The experimental | 0.2397 | 2.2369 | 2.5884 | 2.677 | 3.4389 |
| group | 0.2345 | 2.1553 | 2.415 | 2.3482 | 3.5257 |
| | 0.2426 | 2.2466 | 2.484 | 2.4602 | 3.2981 |
| Mean value | 0.2389 | 2.2129 | 2.4958 | 2.4951 | 3.4209 |
| The control group | 0.3226 | 1.1885 | 1.3966 | 0.4704 | 1.8101 |
| (Taxol) | 0.3239 | 1.1468 | 1.3211 | 0.451 | 1.8912 |
| | 0.3176 | 1.2843 | 1.5144 | 0.4672 | 1.9201 |
| Mean value | 0.3214 | 1.2065 | 1.4107 | 0.4629 | 1.8738 |
| The untreated group | 0.3018 | 0.8339 | 0.5024 | 0.5019 | 2.0492 |
| | 0.2869 | 1.1646 | 0.5128 | 1.7542 | 1.987 |
| Mean value | 0.2944 | 0.9993 | 0.5076 | 1.1281 | 2.0181 |
| The experimental | | 1.9740 | 2.2569 | 2.2562 | 3.1820 |
| group - Blank | | | | | |
| The control group | | 0.8852 | 1.0893 | 0.1415 | 1.5524 |
| (Taxol) - Blank | | | | | |

The viability

| | | | | |
|---|---|---|---|---|
| The | 1.9755 | 4.4462 | 2.0001 | 1.5767 |
| experimental | | | | |
| group | | | | |
| The control | 0.8858 | 2.1460 | 0.1254 | 0.7693 |
| group | | | | |
| (Taxol) | | | | |

The cytotoxicity

| | | | | |
|---|---|---|---|---|
| The | −0.9755 | −3.4462 | −1.0001 | −0.5767 |
| experimental | | | | |
| group | | | | |
| The control | 0.1142 | −1.1460 | 0.8746 | 0.2307 |
| group | | | | |
| (Taxol) | | | | |

From the above data, it is clear that the viability of normal cell is close to 1, or more than 1, and the cytotoxicity against normal cell is close to 0.

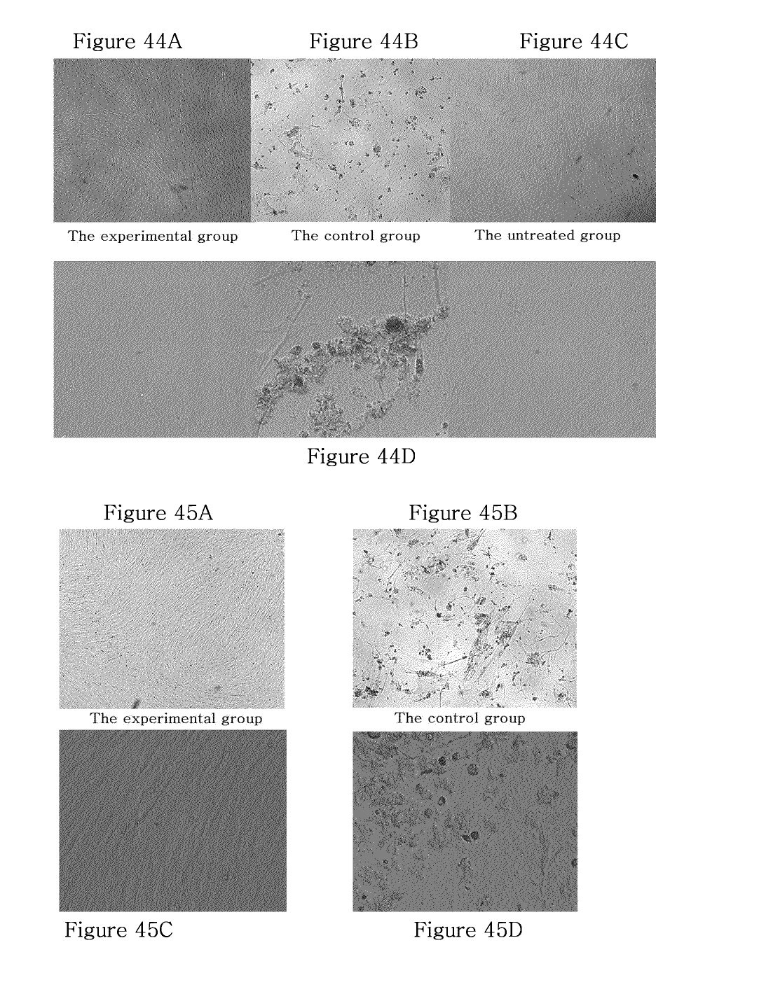

In addition, it was observed that cells in the experimental group were well-grown similar to that in the untreated group, whereas cells in the control group treated with Taxol were fully destroyed (FIGS. 44 and 45).

(12) CCD-1074sk

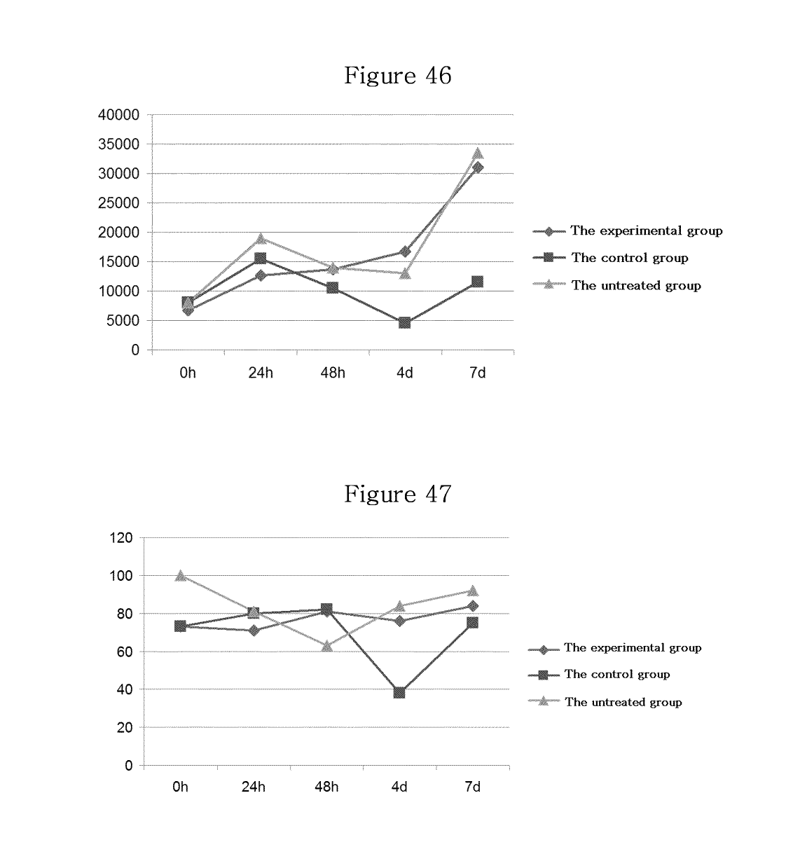

The cell counts is shown in the following Table 34 and the viability is shown in the following Table 35, after determined in cell line CCD-1074sk during the 1^{st} week (LC50) (FIGS. 46 and 47). The viability was determined by using the trypan blue staining in the well of cell counting.

TABLE 34

| | 0 h | 24 h | 48 h | 4 d | 7 d |
|---|---|---|---|---|---|
| The | 7000 | 7000 | 11000 | 25000 | 27000 |
| experimental | 4000 | 13000 | 15000 | 17000 | 25000 |
| group | 9000 | 18000 | 15000 | 8000 | 41000 |
| Mean value | 6667 | 12667 | 13667 | 16667 | 31000 |
| The control | 9000 | 15000 | 10000 | 6000 | 11000 |
| group (Taxol) | 7000 | 16000 | 11000 | 3000 | 12000 |
| Mean value | 8000 | 15500 | 10500 | 4500 | 11500 |
| The untreated | 10000 | 13000 | 11000 | 12000 | 24000 |
| group | 6000 | 25000 | 17000 | 14000 | 43000 |
| Mean value | 8000 | 19000 | 14000 | 13000 | 33500 |

TABLE 35

| | 0 h | 24 h | 48 h | 4 d | 7 d |
|---|---|---|---|---|---|
| The | 100 | 50 | 100 | 76 | 78 |
| experimental | 50 | 73 | 72 | 84 | 82 |
| group | 70 | 90 | 71 | 67 | 92 |
| Mean value | 73 | 71 | 81 | 76 | 84 |
| The control | 70 | 71 | 80 | 43 | 92 |
| group (Taxol) | 75 | 89 | 83 | 33 | 57 |
| Mean value | 73 | 80 | 82 | 38 | 75 |
| The untreated | 100 | 80 | 63 | 79 | 89 |
| group | 100 | 82 | 63 | 88 | 94 |
| Mean value | 100 | 81 | 63 | 84 | 92 |

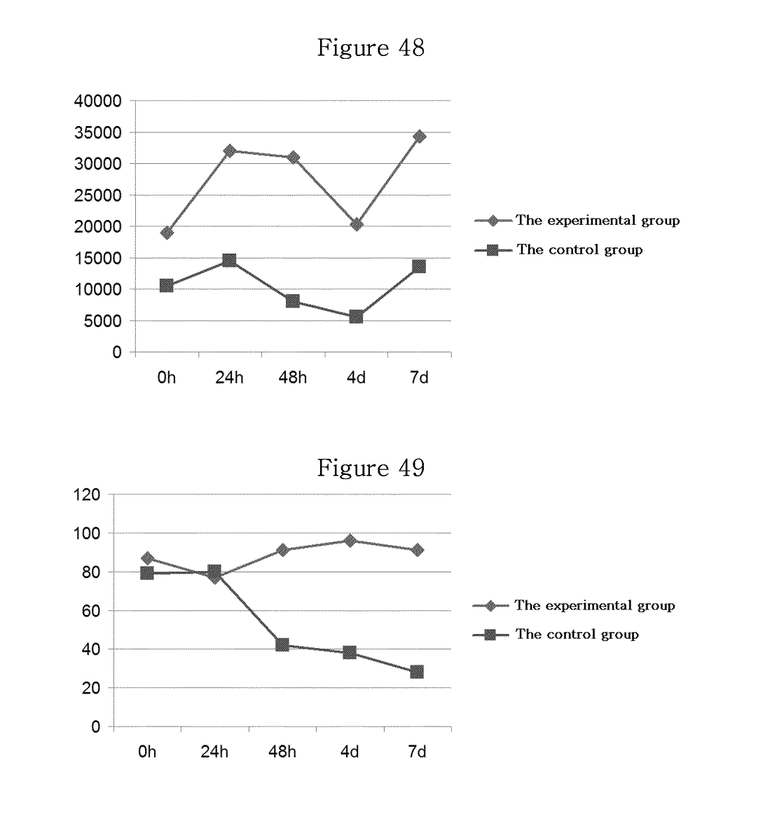

The cell counts is shown in the following Table 36 and the viability is shown in the following Table 37, after determined in cell line CCD-1074sk during the 2^{nd} week (recovery) (FIGS. 48 and 49). The viability was determined by using the trypan blue staining in the well of cell counting.

TABLE 36

| | 0 h | 24 h | 48 h | 4 d | 7 d |
|---|---|---|---|---|---|
| The | 21000 | 25000 | 31000 | 19000 | 41000 |
| experimental | 18000 | 39000 | 29000 | 20000 | 33000 |
| group | 18000 | 32000 | 33000 | 22000 | 29000 |
| Mean value | 19000 | 32000 | 31000 | 20333 | 34333 |
| The control | 10000 | 8000 | 10000 | 4000 | 18000 |
| group (Taxol) | 11000 | 21000 | 6000 | 7000 | 9000 |
| Mean value | 10500 | 14500 | 8000 | 5500 | 13500 |

TABLE 37

| | 0 h | 24 h | 48 h | 4 d | 7 d |
|---|---|---|---|---|---|
| The | 88 | 79 | 100 | 96 | 87 |
| experimental | 87 | 68 | 88 | 96 | 91 |
| group | 85 | 84 | 85 | 96 | 94 |
| Mean value | 87 | 77 | 91 | 96 | 91 |
| The control | 82 | 71 | 27 | 43 | 19 |
| group (Taxol) | 75 | 89 | 57 | 33 | 37 |
| Mean value | 79 | 80 | 42 | 38 | 28 |

### Example 7

### In Vivo Experiments

In Vivo Anticancer Effects

WM-266-4 (human melanoma) was selected as cancer cell line. PBS and 1×10⁵ cells were mixed with Matrigel (BD biosciences) in a weight ratio of 1:1 to obtain the mixture solution containing WM-266-4 cells. Each of fourteen 5-week-old female nude mice (BALB/c nu/nu) was inoculated to the right flanks with 80 μl of the mixture. And then, when the mean value of tumor burden became 1000, any agents were not administered to five mice (the untreated group), cisplatin 0.0025 mg/g (weight of compound/body weight of mouse) were administered to other five mice (the control group), and the compound of the formula II and III 0.00035 mg/g (weight of compound/body weight of mouse) were separately administered to other four mice twice a day (the experimental group).

Tumor burden=π/6×0.5×length×(width)².

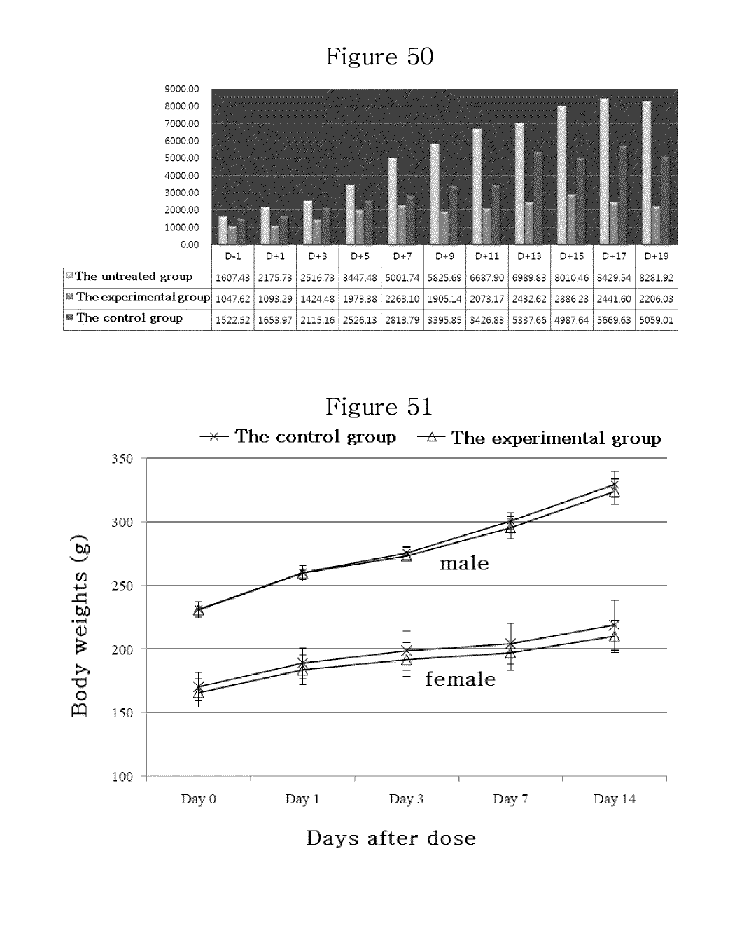

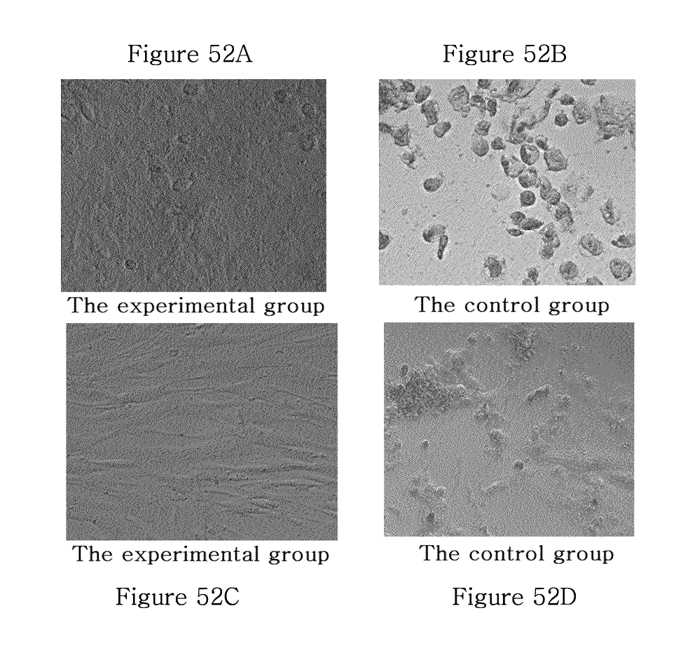

The size of tumors and the body weight of mice were measured every 2 days. After mice were sacrificed, the size of tumors removed and the body weight of mice were measured. The result is shown in FIG. 50.

It was observed that the size of tumor in the experimental group was remarkably smaller than that in the control group.

(2) Differentiation of T Lymphocytes in Nude Mice

PBS was orally administered to three 5-week-old female nude mice (BALB/c nu/nu), and the compound of the formula II according to the invention was orally administered to the other three 5-week-old female nude mice (BALB/c nu/nu) at dose level of 0.00033 mg/g (weight of compound/body weight of mouse) twice a day for 18 days.

FACS analysis was performed to identify the number of T lymphocytes in the Peripheral Blood Mononuclear Cells (PBMC) of nude mice. Anti-mouse CD8 was used as primary antibody, and FITC-conjugated rat anti-mouse IgG was used secondary antibody.

The result is shown in the Table 38. The number of T lymphocytes in the experimental group is twice as many as the number in the control group treated with PBS, and is close to the number in wild type rats.

The result demonstrates that the composition according to the invention induced adult stem cells to differentiate into T lymphocytes, since the increase in T lymphocytes population can occur only through stem cell differentiation in nude mouse without thymus.

TABLE 38

| CD8-positive T lymphocyte % | PBMC |
|---|---|
| Wild type rat | 4.92 |
| Nude mouse treated with PBS | 1.94 |
| Nude mouse treated with the composition | 3.84 |
| according to the invention | |

### Example 8

### Whether to Show Side Effects

In Vivo Toxicity Test in Rats

The present experiment was carried out to evaluate the single-dose oral toxicity of the composition according to the invention, in Sprague-Dawley rats.

The composition according to the invention was administered to male and female rats at dose level of 80 ml/kg (volume of composition/body weight of rat), i.e. 28 mg/Kg (weight of compound/body weight of rat). Vehicle control groups treated with distilled water were set up. Each group was consisted of 5 rats of each sex. Mortalities, clinical signs and body weight changes were monitored for 14 days. At the end of 14-day observation period, all animals were sacrificed and necropsy findings were observed. The results are as follows:
1) No dead animals were observed during the experimental period.
2) No abnormal clinical signs were observed.
3) There were no notable test article-related changes in body weight (FIG. 51).
4) No test article-related abnormal gross findings were observed.

(2) Hairs in Nude Mice

The composition according to the invention was orally administered to 5-week-old female nude mice (BALB/c nu/nu) at dose level of 0.00033 mg/g (weight of compound/body weight of mouse) twice a day for 2 weeks. The group was consisted of 10 nude mice.

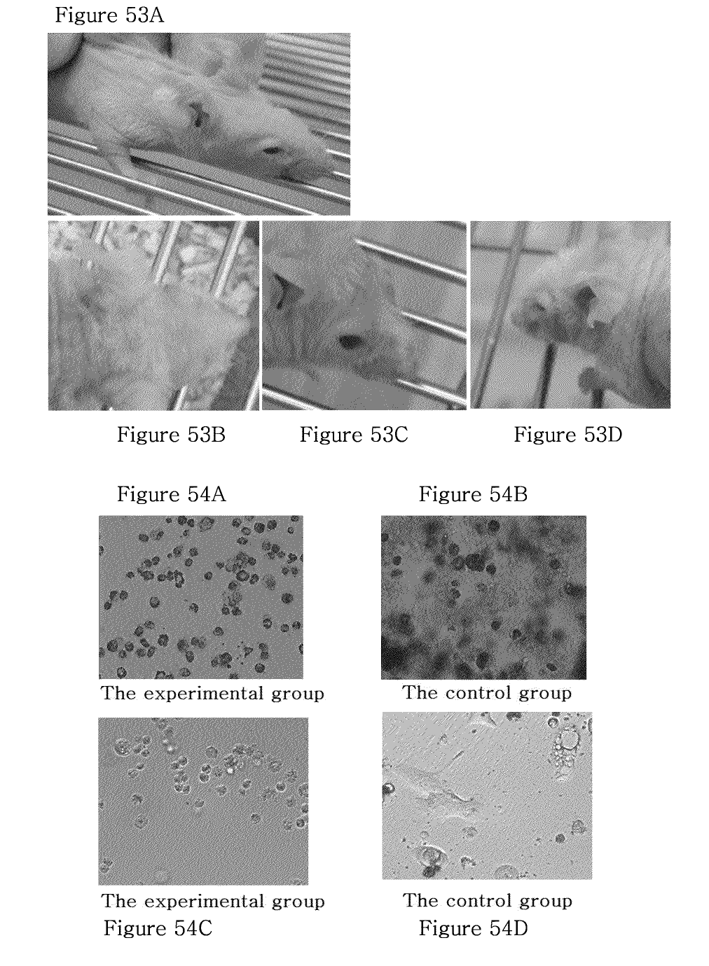

As a result, it is found in all of the hairless nude mice that their hair started to grow on heads and flanks (FIG. 53). This phenomenon means that abnormal genes in nude mice, after being treated with the composition according to the invention, changed into normal ones in wild type mice having white hair.

Therefore, the composition according to the invention as an anticancer agent does not have side effects of hair loss.

(3) The Form After Cancer Cells are Dead

All cancer cells tested in vitro, regardless of their expression of molecular markers, were dead in the unique way by the composition according to the invention. It kills cancer cells in relatively cleaner way than Taxol (FIG. 54).

Therefore, the composition according to the invention can be suitably used to cancer patients with weakened body functions since the burden to eliminate the residues is reduced within their bodies.

What is claimed is:

1. A compound of the formula I: 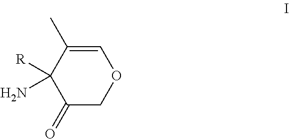
wherein R is C₂H₅ or C₂H₃,
or a pharmaceutically acceptable salt thereof.

2. A pharmaceutical composition for treating or preventing a cancer, comprising the compound of the formula I or a pharmaceutically acceptable salt thereof according to claim 1 as an active ingredient.

3. The pharmaceutical composition according to claim 2, wherein said cancer is breast cancer, prostate cancer, lung cancer, liver cancer, pancreatic cancer, skin cancer, colorectal cancer, osteosarcoma or brain tumor.

4. The pharmaceutical composition according to claim 2, wherein the composition is administered by parenteral, topical, oral, rectal or nasal route.

5. A process for preparing the compound of the formula I according to claim 1, comprising:
a) Reacting (i) 2-methyl-butyric acid and (ii) arctigenin-4-O-glucoside, 3,5,7,9-tetrayne or alanine, in a weight ratio of 2:1;
b) Adding (i) copper and (ii) luteolin-7-rhamnoglucoside, saponin, pinene, trans-geraniol, linalool or chlorogenic acid in a weight ratio of 3:1 to the products obtained in the step a), and then reacting;
c) Adding (i) vitexicarpin or hesperidin and (ii) 3′-hydroxyformononetin, kaempferol or water in a weight ratio of 1:1 to the products obtained in the step b), and then reacting;
d) Reacting the products obtained in the step c) and sinigrin; and
e) Reacting the products obtained in the step d) and valine to obtain the compound of the formula I.

6. The process according to claim 5, wherein the reacting in the step a) is carried out at a temperature of 80˜120° C. for 10˜30 minutes.

7. The process according to claim 5, wherein the reacting in the step b) is carried out at a temperature of 120˜170° C. for 10 minutes.

8. The process according to claim 5, wherein the reacting in the step c) is carried out at a temperature of 80˜120° C. for 5˜8 minutes.

9. The process according to claim 5, wherein the reacting in the step d) is carried out at a temperature of 100˜140° C. for 5˜10 minutes.

10. The process according to claim 5, wherein the reacting in the step e) is carried out at a temperature of −30˜30° C. for 10˜20 minutes, or at a temperature of 80˜230 for 5˜30 minutes.

11. The process according to claim 10, wherein the reacting in the step e) is carried out at a temperature of −30˜30° C. for 10˜20 minutes, and then at a temperature of 80˜230° C. for 5˜30 minutes.

12. The process according to claim 5, wherein water (H₂O) is used as the solvent in the step a) to the step e).

13. The process according to claim 5, wherein the products obtained in the step e) are filtered by water at −5˜30° C.

14. The process according to claim 5, wherein the products obtained in the step e) are filtered by water at 100˜150° C.

15. The process according to claim 14, wherein the filtered products are further filtered by water at 70˜100° C.

16. The process according to claim 15, wherein the filtered products are further filtered by water at 40˜60° C.

17. The process according to claim 16, wherein the filtered products are further filtered by water at 15˜30° C.

18. The process according to claim 17, wherein the filtered products are further filtered by water at −1˜15° C.

19. The process according to claim 14, wherein the filtered products are further filtered by water at 30˜100° C.

20. The process according to claim 19, wherein the filtered products are further filtered by water at −5˜30° C.

21. The process according to claim 13, wherein the filter used in said filtering is the membrane with a pore size of not bigger than 10⁻⁶ m.

22. The process according to claim 14, wherein the filter used in said filtering is the membrane with a pore size of not bigger than 10⁻⁶ m.

23. The process according to claim 15, wherein the filter used in said filtering is the membrane with a pore size of not bigger than 10⁻⁶ m.

24. The process according to claim 16, wherein the filter used in said filtering is the membrane with a pore size of not bigger than 10⁻⁶ m.

25. The process according to claim 17, wherein the filter used in said filtering is the membrane with a pore size of not bigger than 10⁻⁶ m.

26. The process according to claim 18, wherein the filter used in said filtering is the membrane with a pore size of not bigger than 10⁻⁶ m.

27. The process according to claim 19, wherein the filter used in said filtering is the membrane with a pore size of not bigger than 10⁻⁶ m.

28. The process according to claim 20, wherein the filter used in said filtering is the membrane with a pore size of not bigger than 10⁻⁶ m.

29. A method of treating or preventing cancer in a human or animal subject, comprising administering to the subject a compound of formula I 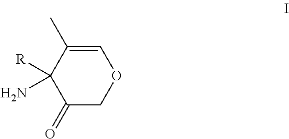
wherein R is C₂H₅ or C₂H₃,
or a pharmaceutically acceptable salt thereof.

30. The method according to claim 29, wherein said cancer is breast cancer, prostate cancer, lung cancer, liver cancer, pancreatic cancer, skin cancer, colorectal cancer, osteosarcoma or brain tumor.

31. The method according to claim 29, wherein the composition is administered by parenteral, topical, oral, rectal or nasal route.
